(19) United States
(12) Patent Application Publication
(10) Pub. No.: US 2009/0069642 A1
(43) Pub. Date: Mar. 12, 2009

(54) Wearable Wireless Electronic Patient Data Communications and Physiological Monitoring Device

(75) Inventors: Tia Gao, Ellicott City, MD (US); Joel Selanikio, Washington, DC (US); Leo Selavo, Riga (LV)

Correspondence Address: PATENTBEST, 4600 ADELINE ST., #101, EMERYVILLE, CA, 94608, US

(73) Assignee: AID Networks, LLC, Rockville, MD (US)

(21) Appl. No.: 12/208,540

(22) Filed: Sep. 11, 2008

Related U.S. Application Data
(60) Provisional application No. 60/971,516, filed on Sep. 11, 2007.

(51) Int. Cl. A61B 5/00; G06Q 50/00

(52) U.S. Cl. 600/300; 705/2

(57) ABSTRACT

Described are patient data communication devices that may be used as wearable patient monitors. The devices are adapted to accept essentially any type of data from essentially any data source, and are reconfigurable, such that each device can determine which data inputs and outputs should be active, and can reconfigure itself based on new configuration instructions. The devices include wireless transceiver units that allow them to form networks, and particularly mesh networks, with other devices. In a mesh network, any one of the devices may serve as a data source, a data forwarder, or a data sink, and the processor of each device may determine whether data should be outputted, displayed, or processed on the local device or on a remote device in the network. Data from other devices in a mesh network may be accepted selectively, depending on the number of hops between the sending and receiving devices.

### CROSS-REFERENCE TO RELATED APPLICATIONS

This application claims priority to, and the benefit of, U.S. Provisional Patent Application No. 60/971,516, filed Sep. 11, 2007, the contents of which are incorporated by reference in their entirety.

### STATEMENT REGARDING FEDERALLY-FUNDED RESEARCH AND DEVELOPMENT

This invention was made, in part, with funds provided by the Department of Homeland Security SBIR Program under Contract No. NBCHC080059. The U.S. Government may have certain rights in this invention.

### BACKGROUND OF THE INVENTION

1. Field of the Invention

This invention relates generally to the field of medical monitors and sensors, and more particularly to wireless medical monitors.

2. Description of Related Art

Patient treatment management has increasingly relied on electronic monitoring. A growing number of medical devices, sensors and monitors are used to track a patient's condition and to aid in patient treatment. For example, sensors may provide data on Electrocardiogram (ECG), electroencephalogram (EEG), heart rate, blood pressure, pulse oximetry, body temperature, blood chemistry and other vital signs and indicators, which may be used as diagnostic tools, to treat a patient, and to allocate medical resources to patients requiring care.

The majority of these devices are stand-alone devices, which do not communicate with other devices, or with a central station that may be easily reviewed by a medical professional. Traditional patient monitoring has involved connecting a patient to one or, more likely, many different medical devices. These devices may be connected to bedside monitors, which may take up a great deal of space, and limit the patient's mobility, as well as access to the patient. Although size of many of these devices has been decreasing, the number of monitors used on a patient has been increasing.

Thus, there is a need to consolidate the presentation, control, and monitoring of patient data, such as by presenting all information from patient-related devices (including patient vital signs and other relevant patient information) together, and to allow centralized control and monitoring of any medical devices that are connected to a patient. Achieving consolidated presentation, control, and monitoring of patient data is believed to be complex and difficult, because individual patients may use a different array of medical devices, which may not readily interconnect.

There is also a need to provide a wearable device which may centralize monitoring and control of other medical devices connected to a patient. A wearable monitoring device that is adaptable or configurable to each patient's monitoring needs could achieve this and provide previously unrealized flexibility.

### SUMMARY OF THE INVENTION

One aspect of the invention relates to a patient information communication device. The patient information communication device comprises one or more patient data inputs, one or more patient data outputs, and a processor. Each of the one or more patient data inputs is adapted to accept data from one or more medical sensors or actuators. The processor is connected to the one or more patient data inputs and the one or more patient data outputs. The processor is adapted (1) to determine, based on a set of configuration instructions, which of the one or more patient data inputs and which of the one or more patient data outputs should be active; (2) to determine the manner and type of the data that should be outputted to the one or more patient data outputs; and (3) to accept new configuration instructions, either explicitly or as a result of a change in the nature of the one or more medical sensors or actuators, and dynamically reconfigure the patient information communication device based on the new configuration instructions. The patient information communication device is wearable.

Another aspect of the invention also relates to a patient information communication device. The patient information communication device comprises a wireless transceiver unit and a processor. The wireless transceiver unit is configured and adapted to input and output data. The processor is connected to the wireless transceiver unit and adapted to configure the wireless transceiver unit to allow the patient information communication device to act as a node in a mesh network. The processor and wireless transceiver unit are operable to allow the device to perform one or more network functions selected from the group consisting of data source, data forwarder, and data sink.

Yet another aspect of the invention relates to a system for communicating and managing patient information. The system comprises a plurality of patient information communication devices. Each of the devices includes a wireless transceiver unit and a processor. The wireless transceiver unit is configured and adapted to input and output data. The processor is connected to the wireless transceiver unit and adapted to configure the wireless transceiver unit to allow the patient information communication device to act as a node in a mesh network. The processor and wireless transceiver unit are operable to allow the device to perform one or more network functions selected from the group consisting of data source, data forwarder, and data sink. At least some of the plurality of patient information communication devices are wearable. The plurality of patient information communication devices are configured to establish a mesh network with one another. At least some of the plurality of patient information communication devices further comprise additional hardware components including processing components, memory components, or user-interface components, allowing those devices to perform one or more of storing data, forwarding data, presenting data, or modifying and annotating data for others of the plurality of patient information communication devices.

Other aspects, features, and advantages of the invention will be set forth in the description that follows.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1A is a schematic diagram of one embodiment of a dynamically reconfigurable patient information communication device as described herein;

FIG. 1B is a schematic diagram of another embodiment of a dynamically reconfigurable patient monitor;

FIG. 2 is flow diagram illustrating one method of monitoring a patient using a dynamically reconfigurable patient monitor;

FIG. 3 is a flow diagram illustrating another method of monitoring a patient using a dynamically reconfigurable patient monitor;

FIG. 4 is a flow diagram illustrating a method of managing a plurality of patient monitors, as described herein;

FIG. 5 is schematic illustration of an ad-hoc mesh network including multimodal patient monitors;

FIG. 6 is a schematic of one embodiment of a multimodal patient monitor; and

FIG. 7 is an information flow diagram of one example of a dynamically reconfigurable patient monitor as described herein.

### DETAILED DESCRIPTION OF THE INVENTION

Described herein are patient information communication devices and methods of using them. In some embodiments, the communication devices serve as patient monitors; therefore, in this description, the term “patient monitor” may be used to mean “patient information communications device.” Also described herein are methods of monitoring a plurality of patients each wearing a patient monitor. Patient information communication devices according to some embodiments of the invention may also be reconfigured to act as nodes on a communication network and to serve as monitors, adapters for other devices, gateways, repeaters, and routers. More generally, patient monitors according to embodiments of the invention serve as communication platforms for patient data, collecting all care-relevant forms of data and communicating that data in ways that will be described below in more detail.

Although the specification is described in various sections or parts, it should be understood that any of the sections described herein may be used or incorporated into any of the descriptions of the various devices and methods, unless the context indicates otherwise.

### Dynamically Reconfigurable Wearable Patient Monitors

A dynamically reconfigurable wearable patient monitor may be worn by a patient before, during, and after patient treatment. The dynamically reconfigurable patient monitor (or “reconfigurable patient monitor” or “monitor”, for short) may be used as a multifunctional patient monitor device. A reconfigurable patient monitor may act as a patient-specific hub that collects as much relevant data as possible. Once collected, this patient data can be analyzed to adjust the parameters of data collection, adjust the parameters of data transmission, trigger one or more patient alerts, it can be presented to a caregiver, or it can be transmitted to a server or processor where further analysis can occur.

In general, a patient monitor according to an embodiment of the invention is competent to receive many different types of patient data, but can be dynamically reconfigured to select the type of patient data information received by the monitor, the way in which the monitor receives that patient data and the way that the patient data is handled. Put another way, a reconfigurable patient monitor may be initially configured to monitor a specific subset of patient data “channels,” where each channel is monitored at a specified monitoring parameters; later the same reconfigurable patient monitor may receive instructions to change its configuration and monitor a second subset of patient “channels” at different monitoring parameters. Reconfiguration instructions may be received by the monitor (e.g., remotely or directly), or they may be based on the on a conditional met by the patient data being monitored.

Thus, a reconfigurable patient monitor may be used to track a patient's progress and current condition (e.g., to monitor vitals), to track their treatment (e.g., prescriptions, diagnosis, etc.), to track and control treatment by one or more treatment devices (e.g., dialysis machines, infusion pump, IV-PCA, or any other bedside patient treatment device), to track additional patient conditions monitored by another monitoring device, to track the patient's physical location (e.g., by GPS), to receive and store caregiver inputs (e.g., text comments, voice comments, image inputs, etc.), and to collect data on workflow processes (e.g., a disaster casualty has passed through the decontamination tent, a resident of a refugee camp has collected rations at the food tent, a rehabilitation patient has done the exercises in a rehabilitation routine, etc.). In cases in which workflow process information is collected and reported, that information may be collected either by an explicit data entry indicating that a patient has done something or a step in a workflow process has been completed, or indirectly, based on other data that is collected. For example, if a medical professional enters triage data for a certain patient, then the monitor may report that the patient has been through the triage area.

Moreover, the same device can be configured to perform only a desired subset of the above tasks, and the device can be reconfigured to adapt to the changing patient needs or physician/health care provider needs. In some device embodiments this is achieved by the use of monitor configuration instructions that can be received and executed by the reconfigurable monitor, as described in greater detail below.

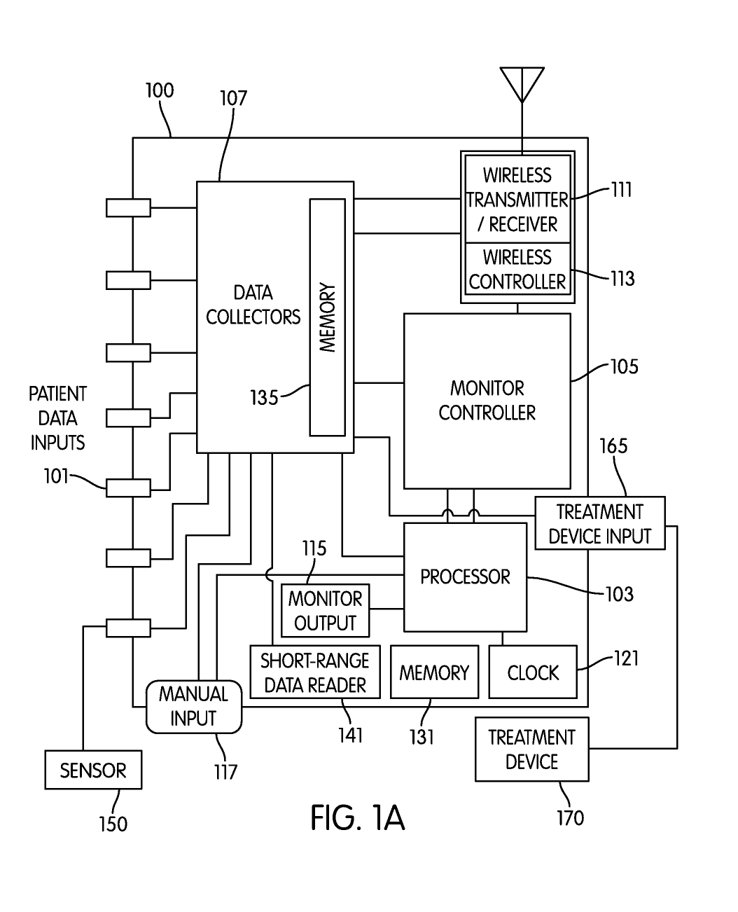

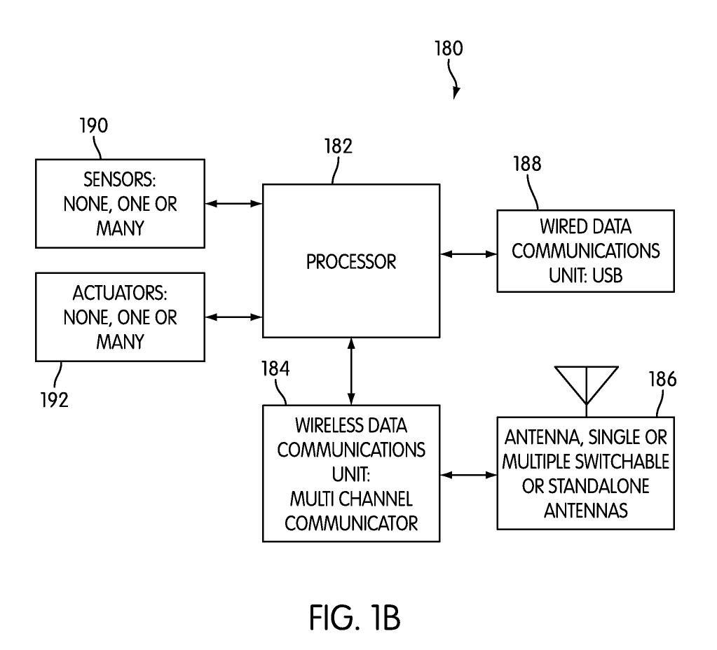

FIG. 1A schematically illustrates some of the components of a first embodiment of a reconfigurable patient monitor 100. These components will be described briefly below, and then examples of these devices will be provided thereafter. The reconfigurable patient monitor in FIG. 1A includes a wireless transmitter/receiver 111, a plurality of patient data inputs 101, a monitor controller 105, a plurality of data collectors 107, a processor 103, and a monitor output 115.

The patient data inputs 101 of FIG. 1A are connected to the data collectors 107. Any appropriate data input may be used. For example, a data input may be an input for a sensor 150 (e.g., a pulse oximeter, an ECG, heart rate sensor, an EEG, a blood pressure sensor, a temperature sensor, a CO₂ sensor, a respiration sensor, a glucose sensor, a skin resistance sensor, anemia detector, hydration sensor, radio frequency ID sensors, global positioning system transceivers, gyroscopes, and accelerometers, digital cameras, IR cameras, ambient humidity sensors, ambient temperature sensors, ambient light sensors, ambient vibration sensors, etc.), particularly sensors to measure patient vital signs. The data inputs may be physical inputs (e.g., plugs) that are configured to mate with a sensor. A data input may also be a wireless data input. For example, a data input may be connected to the wireless transmitter/receiver 111, so that data can be received through the wireless transmitter/receiver. A patient data input may also be a dedicated manual data input, such as a keypad, knob, touch-screen, button, handle, dial, etc. Some manual data inputs are located on the surface of the device (e.g., the housing) and may be manipulated by a patient or health care provider to input data into the monitor.

In some embodiments the patient data input is reconfigurable. For example, the same keypad or button may be used to input different patient data. A device output (e.g., display) may be coordinate with one or more patient data inputs, to indicate what patient data is being read by a particular patient data input. This may be accomplished with a programmable touch screen, for example.

A patient data input may also be a keypad or numeric touchpad, which may accept text or numeric patient-related information. In some embodiments the patient data input is dedicated to receive a particular type of input (e.g., it may fit a particular shape of plug or may be specific to a certain type of patient monitoring device).

One class of data inputs are short-range data readers 141 (also referred to as “short-range electronic data readers”). Short range data readers may include RFID readers, barcode scanners, smart card readers, fingerprint readers, and the like. A short range data reader may be used as one type of patient data input (e.g., for inputting information about medication taken, caregiver attention, etc.). A short range data reader may also have uses other than patient data collection. For example, a short range data reader may act as a security feature to prevent unauthorized use of the monitor. Thus, operation of the device (e.g., to activate the device, to manually input patient data, to display patient data, to change the operational mode of the device, etc.) may be gated by a short-range data reader such as a fingerprint reader or card reader.

In some embodiments, a patient data input is a treatment device input 165. Thus, patient data may be received from a treatment device 170 such as a stimulator (e.g., electrical stimulator), an infusion pump, an IV-PCA, etc. The treatment device input may also be a treatment device output through which the monitor may pass control signals.

A reconfigurable patient monitor typically includes a plurality of patient data inputs, as indicated schematically in FIG. 1A, and may have both dedicated data inputs (dedicated to a particular type of data input such as temperature, ECG, etc.) and reconfigurable data inputs. As indicated in FIG. 1A, the patient data inputs 101 are connected to data collector 107.

A data collector 107 may be connected to a data input 101. In general, data collectors control the collection of data from the patient data inputs. For example, a data collector may include a control for turning on/off the data collection from a particular data input, a control for sampling the data input, a control to determine if the input is connected or operational, etc. A data collector may also include a memory 135 for storing data collected from the data input. In some embodiments, the data collector includes a register or buffer for holding data collected from a patient data input. A particular data collector may be dedicated to a particular data input, or a data collector may be configurable to connect to all or a subset of patient data inputs. The data collectors 107 may be controlled by the monitor controller 105, which can dynamically determine which data collectors (and, correspondingly, which patient data inputs) are active.

As mentioned, the monitor controller includes monitor configuration instructions that select which patient data inputs are active and what the patient data input parameters are. In operation, the monitor controller 105 activates data collectors 107 corresponding to the patient data inputs 101 indicated by the monitor configuration instructions, and causes the data collectors 107 corresponding to each of the indicated data inputs 101 to operate using the configuration instructions for that data input.

Although a reconfigurable patient monitor may control which patient data inputs 101 are actively monitored at any given time by configuring the data input parameters (based on the monitor configuration instructions), in some embodiments, one or more of these patient data inputs are continuously active to receive patient data, regardless of the monitor configuration instructions. For example, sensor input from a heart rate detector may always be active, allowing continuous monitoring of the patient, while a caregiver input (e.g., voice input by a caregiver) may be activated on demand, thus allowing a caregiver to provide annotations to a patient monitor when they need to.

The data collectors 107 may also be connected to a processor 103. A processor 103 may process all or a subset of the collected patient data from the data collectors 107. In particular, the processor may apply patient data alert parameters provided by the monitor configuration instructions in the monitor controller 105. Thus, the processor may compare the collected data to the corresponding patient data alert parameter; if the collected data indicates is within the alert parameter, the processor may signal an alert (e.g., by communication with a monitor output 115, or by wireless signal through the wireless transmitter/receiver 111, or by informing the monitor controller 105 to submit alert information with the collected data.).

The processor 103 may also be used to at least partially analyze the collected data to determine one or more indicators of general patient status, even when an alert is not triggered. The processor may locally (at the level of the wearable patient monitor) display patient status indicators from the data collected (e.g., heart rate, ECG, temperature, pain level, etc.) via a monitor output 115. The processor 103 may also prepare the data for transmission via wired or wireless connection to an external device (e.g., an external client, a server, etc.). Data collected by the data collectors may be time-stamped (using the clock 121), condensed, encrypted, parsed, coded for error correction, and/or marked with a device- or patient-identifier. In some embodiments, this step may occur at locations other than the processor 103 (e.g., at the controller 105 or the wireless controller 113.)

Data collectors 107 may also connected to the monitor controller so that the monitor controller can regulate the export of collected data via the wireless transmitter/receiver. In some embodiments, the data collectors 107 are directly connected to the wireless transmitter/receiver (or the wireless controller 113), for transmitting the data (or a portion of the data).

The wearable patient monitor may also include a device memory 131 for storing patient and/or monitor information. Thus, if the device is not in contact with a network (or otherwise connected to an external client or server), it may store patient data until a connection is made, or until a manual download of the monitored information is performed. Thus, the memory may be connected to the data collectors. The memory may also be used to record information about the status of the monitor, such as when it was accessed, any error codes generated, or the like.

The elements illustrated may be arranged differently, and may be connected in a variety of different ways, including connections not indicated in FIG. 1A. FIG. 1A is not intended to be a complete schematic, and a wearable patient monitor 100 may include additional features or elements that are not shown. For example, the device may include built-in sensors.

Although the description above refers to data collectors 107 connected to data inputs 101, a processor 101, and device memory 131, in some embodiments, one component may perform multiple functions, and in other embodiments, the functions ascribed to one component may be performed by several components. FIG. 1B is a schematic illustration of another embodiment of a reconfigurable patient monitor 180 illustrating this principle. The patient monitor comprises a processor 182 that may, for example, handle digital signal processing, data processing, and communications scheduling. The processor 182 communicates with a wireless data communications unit 184, which may be a multi-channel communicator. The wireless data communications unit 184 is, in turn, connected to an antenna or antennas 186. The antenna 186 may be a single antenna, multiple switchable antennas, or standalone antennas. The patient monitor 180 may optionally also include a wired data communications unit 188, which may, for example, be a universal serial bus (USB) port. The patient monitor 180 may be connected to any number of sensors, which are indicated collectively by reference numeral 190, and may also be connected to any number of actuators, indicated collectively by reference numeral 192.

In the foregoing description, general terms such as “processor” and “memory” are used. These terms should be construed to refer to any devices that are capable of performing the described functions. The processor, for example, may be a microprocessor, an ASIC, or any other similar type of device. The memory may be random access memory (RAM), read-only memory (ROM), programmable read-only memory, flash memory, etc. Several components may be integrated into the same package or chip. For example, in one embodiment, a patient monitor 100, 180 may use a Texas Instruments CC2430, which combines a processor and wireless data communications unit. In another embodiment, a patient monitor 100, 180 may use a Texas Instruments TI MSP 430 processor with a CC2420 as a wireless data communications unit.

As mentioned, the reconfigurable patient monitors are wearable patient monitors. In some embodiments, the reconfigurable patient monitors are at least partially enclosed in a housing. The housing may protect and help organize the workings of the device. The device may be small enough so that it can be comfortably worn by a patient. For example, the device may be worn around the subject's neck, around a leg or arm (e.g., as a wrist strap), around the abdomen, or the like. In some embodiments, the device includes a means of securing the device to the patient, such as a strap, belt, clip, adhesive, or the like. Any appropriate means of securing the device to the patient (or to an article worn by the patient) may be used.

In operation, the reconfigurable patient monitor is worn by a patient to assist in the medical care of that patient. As mentioned above, the reconfigurable patient monitor may aggregate and funnel patient data from a variety of sources and store the data or pass it on to a destination such as a client device or central server. The patient monitor may also process some of the data to determine if an alert should be triggered (e.g., to alert a caregiver) or to provide other output. The reconfigurable, wearable patient monitor may initially be instructed to monitor a first set of data inputs, and later (while still in use by a patient) be reconfigured to change the data inputs being monitored or the way in which the currently monitored data inputs are monitored. This can be accomplished by changing or replacing a set of monitor configuration instructions used by the patient monitor.

In one embodiment, the monitor controller receives a first set of monitor configuration instructions. As mentioned above, monitor configuration instructions typically indicate (1) the set of patient data inputs to be monitored, (2) the attributes for each patient data input to be monitored, and, optionally, (3) monitoring decision rules for at least some of the patient data inputs indicated. The monitor configuration instructions may be selected either locally at the monitor, or remotely (at a client or server). The monitor may have a default set of configuration instructions to which it initially defaults when first activated.

The patient monitor may also be controlled to ‘start’ monitoring, ‘stop’ monitoring, and to ‘report’ patient data. For example, the monitor may be told when to begin monitoring either remotely or by a control on the monitor, and once it is monitoring, may continuously or periodically report the monitored patient data (or a subset of the patient data) by sending it to a client processor or server. During the monitoring period, the monitor may be reconfigured by changing the monitor configuration instructions. New monitor configuration instructions may be input to the device remotely or locally on the device itself. In some embodiments, the monitor controller may alter its own monitor configuration instructions, based on monitor control logic. For example, if one or more of the patient data inputs registers a value that triggers a patient data alert parameter, the monitor control logic may alter the monitor configuration instructions in response. For example, the patient data input parameter for the patient data input(s) triggering the alert may be modified to increase the sampling rate. The monitor control logic may also be modified (e.g., the control logic may be programmed). In another example, the patient monitor alters its monitor configuration instructions based on the location of the patient (e.g., emergency room, operating room, etc.). Thus, the monitor may include a table or menu of preset monitor configuration instructions. A user may manually select configuration instructions from these presets or the monitor control logic may select appropriate instructions based on patient data inputs (including patient data conditions, patient identifying information, patient location, etc.).

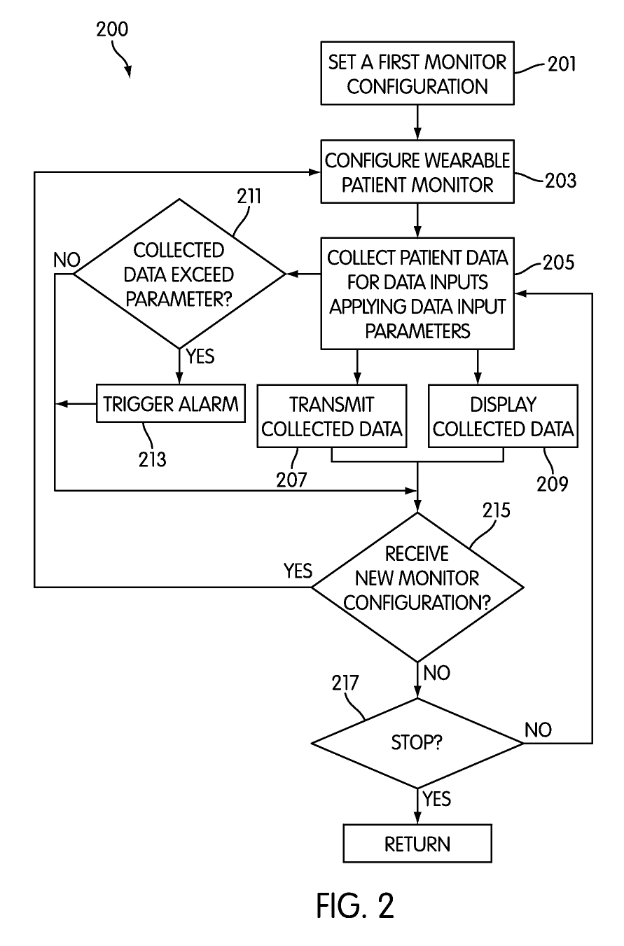

FIG. 2 is a flow diagram illustrating one embodiment of a method, generally indicated at 200, for monitoring a subject using a dynamically reconfigurable patient monitor worn by a patient. In FIG. 2, method 200 begins at task 201 by setting a first monitor configuration. Generally, this is done by providing the monitor controller with a set of monitor configuration instructions, typically including data inputs, data input parameters, and alert parameters. For example, the patient monitor configuration instructions may indicate that the patient monitor should monitor temperature, EEG and should receive patient information (caregiver information). Thus the data inputs would be temperature, EEG, and caregiver information. The monitor configuration instructions may indicate what patient data inputs correspond to these data inputs, or they may rely on a look-up table to determine which data inputs correspond to which physical data inputs in a monitor. Examples of the kinds of data inputs that may be included with a patient monitor are provided in the examples sections below. The data input parameters typically provide instructions for collecting the data for each data inputs. For example, the data input parameter may instruct each data collector how to collect data input from its corresponding data input. Thus the data input parameters may be specific to the data input. Exemplary data input parameters include sample rate, gain, sample block size, conversion (e.g., digital to analog conversion), conversion factors, etc.

Monitor configuration instructions may be input at the wearable patient monitor, or remotely (e.g., from a server or client communicating with the wearable patient monitor). Instructions may be selected from a menu of possible instructions, which may include ‘preset’ or suggested instructions. For example, instructions may be selected based on the treatment regime for the patient, such as emergency room monitoring, diabetic monitoring, burn victim monitoring, coronary disease monitoring, etc. In some embodiments, instruction sets may be based on the location of the patient within the hospital (e.g., patient intake, emergency room, operating room, recovery room, intensive care unit, burn unit, etc.).

Once the monitor configuration instructions are set in task 201, the wearable patient monitor may be configured in task 203 and patient monitoring can then begin. Method 200 then continues with task 205, in which patient data is collected from the data inputs indicated by the patient monitor instructions by applying the patient data input parameters. Based on the monitor configuration instructions, this patient data can be processed, stored, transmitted, as shown by task 207, and/or displayed, as shown by task 209, based at least in part on the patient monitor instructions. Task 211 of method 200 is a decision task—after the data is collected, it is determined whether the data values exceed one of the alert parameters. The alert parameters against which the data values are compared may be the alert parameters set in task 201, or they may be alert parameters built into the device. If the data values do exceed the alert parameters (task 211:YES) an alarm is triggered, as shown at task 213 and method 200 continues with task 215. If the data values do not exceed the alert parameters (task 211:NO), method 200 proceeds directly to task 215.

During the ongoing monitoring of the patient, the device may receive new monitor configuration instructions, as mentioned above, or the monitor control logic may determine that the instructions should be modified. Task 215 is a decision task. If new monitor configuration instructions have been received, or if there is some reason to modify the instructions (task 215:YES), method 200 returns to task 203 and the monitor is reconfigured. Following task 215, method 200 continues with task 217, another decision task. In most embodiments, method 200 will continue until a stop signal is received. In task 217, if a stop signal has been received (task 217:YES), method 200 terminates and returns. If no stop signal has been received (task 217:NO), method 200 returns to task 205 and continues to collect data.

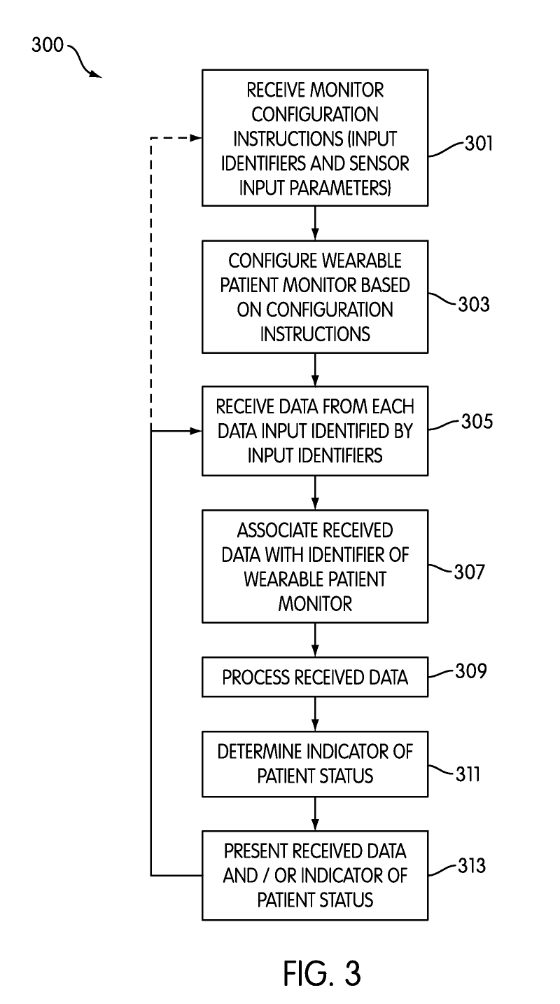

FIG. 3 is a flow diagram illustrating another method 300 of operating a wearable patient monitor. It should be understood that certain tasks of method 300 may be included in method 200, although they are shown separately for convenience in illustration. Tasks 301 and 303 are similar to the corresponding tasks of method 200: monitor configuration instructions are received and the monitor is configured. Task 305 is also similar to task 205—data is collected. In this example, this means that data can be received from each of the data inputs identified by the input identifiers in the instruction set. As shown in task 307, as data is received, it is associated with an identifier for the wearable patient monitor. For example, the identifier may uniquely identify the patient monitor and/or the patient wearing the patient monitor. This permits the collected data (or a portion of the collected data) to be transmitted and be reliably associated with the correct monitor/patient.

Once data has been associated with an identifier, in task 307, it is processed in task 309. Processing may include formatting the data for transmission, extracting patient status information, as shown in task 311, or determining if an alert should be triggered, as discussed above. The indicator of patient status established in task 311 may be a simplified indicator (“normal”, “critical”, etc.), or it may be a condition-specific indicator (“low blood sugar”, “dehydration”, etc.). The indicator of patient status may be determined based on one or more of the monitored patient data inputs. In some embodiments, the indicator of patient status is determined using the processing logic or controller logic. The indicator of patient status may be determined based on data from multiple patient data inputs, or based on data received from a single patient data input.

Once the patient status indicator is established in task 311, patient data and/or the indicator of patient status may be presented in any appropriate manner, as shown in task 313. For example, the patient data, a subset of patient data or the indicator of patient status may be presented visually (on a screen or monitor, indicated by LEDs, etc.), audibly (via a buzzer, tone, synthesized voice, etc.), electronically (by transmission to remote sites), physically (e.g., vibration), or any combination thereof. The patient data and/or indicator of patient status may be presented on the wearable patient monitor or on a remote client or other device, or in more than one location. This could include displaying the patient data or patient status on the wearable patient monitor (e.g., on a screen) and on a screen of a client device receiving information from the monitor. The format with which the patient data and/or the patient status are presented may also be dynamically reconfigurable. For example, the configuration instructions received by the device may include instructions for formatting the data or patient status. Following task 313, method 300 may return to either task 305 and continue receiving data, or it may return to task 301 and receive new monitor configuration instructions, essentially as described above with respect to method 200. The decision tasks that result in a return to either task 301 or task 305 are not shown in FIG. 3, but the flow of method 300 may be assumed to be essentially similar to that of method 200. Once again, upon receipt of a stop signal, method 300 may terminate and return.

Further illustration of the wearable patient monitors that are dynamically reconfigurable are provided in the examples that follow. Any of the features or elements described in the examples may be included as part of any of the devices, systems and methods described herein.

### EXAMPLE 1

### Dynamically Reconfigurable Wearable Patient Monitor

In one example, a patient monitor may be worn by a patient and may function as a universal platform for patient information collection and dissemination. The patient monitor includes inputs and outputs. The patient data inputs include any relevant patient information, such as sensor variables, care provider input variables, patient input variables, treatment device input variables, monitoring device input variable, and server-generated input variables (“variables” refers to sources of data). The data inputs from care providers and the patient may include, for example, vital signs that require subjective analysis such as pain level and/or consciousness level. These variables may be entered via smart card with a pre-defined set of input data, and/or may be entered via manual, free text, or voice entries. For example, the patient monitor may be configured with a touch screen or a button interface for entering a vital sign or data associated with pain level. The inputs from sensors may include, for example, positioning information, location information, vital sign information (e.g., heart rate, ECG, EEG, blood pressure etc.). These variables, in one embodiment, are detected and inputted into a patient monitor via sensors that are physically coupled to the patient monitor.

As noted above, a patient monitor may also receive input from treatment and monitoring devices. In one embodiment, these devices are physically connected to the patient monitor and provide the patient monitor with their respective outputs/read outs. In another implementation, these devices are wirelessly connected to the patient monitor. In particular, the patient monitor can communicate with these devices via a wireless network, which can be transmitted either through a single hop or multiple hops between the sender and receiver. The single hop messaging is frequently used to communicate with devices located within the patient's body area network range, as a safety measure. The multi-hop messaging is frequently used to communicate with servers situated at, for example, a nursing station, which may be far away from the patient.

In either case, the patient monitor receives inputs and processes them accordingly to present an indicator of patient status by generating an output or set of outputs. The outputs may include alerts for the care providers or they may be control settings for treatment and other external devices. The outputs further include a user interface (“UI”) display that renders data and images associated with vital signs. In one implementation, the display can be adjusted based on who is viewing it. In one example, if the patient is viewing the UI, the UI displays a questions/answers screen. In another example, if the nurse is viewing the UI, the UI displays necessary patient data and allows the nurse to input patient assessment data. In return, the patient monitor generates real-time alerts for any assessments that show dangerous levels.

The output may include messages or instructions to another device, such as overhead call lights or a medical device for patient treatment. In one embodiment, the patient monitor monitors the patient's pain level. When the pain level is elevated beyond the treatment threshold, the patient monitor outputs a control message to an IV-PCA pump that is eluding anesthetic dosages to the patient. If the patient's respiration rate falls below a threshold, the patient monitor can send instructions to turn off the IV-PCA, overriding the pain input. The IV-PCA may either be wired or be wirelessly connected to the patient's monitor.

In one embodiment, illustrating how the patient monitor can be used as an electronic chart, a congestive heart failure patient, Norman, goes to the emergency room because he has a fever. Norman checks in at the registration desk, and the registration nurse puts the patient monitor on Norman. The patient monitor is initially set up to monitor 6 vital signs, which are input either manually or by the physiologic sensors. The vital signs that are manually input include the patient's (Norman's) pain level, which the nurse may input from her computer. Alternatively, the nurse may input the pain level directly to the patient monitor. For example, the nurse asks Norman to describe how much pain he is in, on a scale of 0 to 10, and then inputs this number into the patient monitor via a button attached to the patient monitor. Alternatively, the patient monitor can solicit this information directly from Norman, prompting him to enter this data into the patient monitor via a touch screen interface. Other vital signs may be input into the patient monitor via sensors.

In some embodiments, the vital signs may be transmitted to a server for processing. The server can be a globally hosted server that retrieves patient data from a variety of sources. The server analyzes the vital signs, along with the patient data, compares them with a default threshold or a patient specific threshold, and generates one or more alerts, which are transmitted to the patient monitor device and/or to the nurse computer console. Thus, in some embodiments, the patient monitor does not include any patient data alert parameters, although it does indicate the patient data inputs to be monitored and the parameters for monitoring them.

Along these lines, a patient monitor can help doctors to monitor each of several patients in accordance with specific monitoring criteria for that patient. For example, assume there are 20 patients in an intensive care unit. The diseases afflicting each patient may vary, and their doctor must keep track of different conditions, specific to the disease of the patient. For each patient, the clinician typically uses a computer to select a diagnosis criterion (e.g., Infection Criteria, SIRS criteria, Acute Organic Dysfunction Criteria). The computer may be equipped with software that translates treatment criteria into monitoring algorithm parameters and transmits these monitoring parameters to the patient monitor. The patient monitor receives the monitoring parameters and reconfigures its performance parameters and user interface display so it would only prompt the nurse to enter the inputs as required by the diagnosis criteria. As the nurse begins to do vital sign assessments on her patients, the nurse approaches each patient and the patient's patient monitor displays the vital signs that needs to be recorded by the nurse for the prescribed criteria. The nurse enters this data onto the patient monitor and the patient monitor automatically displays diagnosis criteria stored for the clinicians (on the patient monitor and on the remote monitoring consoles).

To further illustrate, assume that among the 20 patients in the intensive care unit are patients Norman and Kelly. Norman is diagnosed with pneumonia and Kelly is diagnosed with sepsis. Their doctor uses different monitoring protocols specific to each disease. For example, Norman's patient monitor may be configured to periodically solicit, from a care provider, information regarding Norman's urine output level and consciousness level to enable determination of CURB-65 score (a score-based diagnosis for pneumonia patients). In contrast, Kelly's diagnosis does not necessarily take into account urine output and consciousness level, and thus Kelly's patient monitor may be configured to monitor other data sensed via the sensors, not including urine output and consciousness level. While at the intensive care unit, Kelly's condition deteriorates and now the proper diagnoses may include Kelly's urine/consciousness level. Therefore, Kelly's doctor may adjust the monitoring conditions of the patient monitor to periodically seek this information from the care provider. The care provider inputs this information into the patient monitor, which transmits them to a remote server for monitoring/processing.

The patient monitor may also contain a radio (e.g., the wireless transmitter/receiver mentioned above) that can function as a node inside a wireless network. This radio may form an ad-hoc wireless network with other devices, including other patient monitors. When data is forwarded through one patient monitor, it can enforce network traffic engineering (e.g., QoS/quality of service). This is described in greater detail below. In one implementation, data from Norman's patient monitor is transmitted at a high priority, because Norman's is experiencing a quickly worsening fever. Patient monitor data from Norman is routed through Kelly's patient monitor, and then to the central station. Since Kelly is doing fine, with normal vital signs, Kelly's patient monitor receives Norman's data, and forwards it on with higher priority. Kelly's lower-priority (lower priority level) data is delayed until the network is less congested.

Traditionally, a nurse had to carry around a clipboard for each patient. The clipboard would include the patient's charts, and her 4 hours recordings of their vital signs (6 vital signs are recorded, as required by The Joint Commission). The nurse may push around a cart that includes vital sign monitoring machine. For example, the nurse takes the patient's pulse and heart rate with a pulseoximeter, then takes the temperature using a temperature probe, and inflates a blood pressure cuff. This can easily take 5 minutes. The nurse writes all this information down on a piece of paper, wipes down the machine with an antiseptic wipe (also a required step by The Joint Commission) and moves onto the next patient. Using the patient monitor described above, the nurse can walk to each patient, without carrying any devices or clipboards. If the patient is already wearing a patient monitor, he simply reads the patient monitor, and enters any information into the patient monitor that is not already monitored. In the current Joint Commission requirements, the nurse only has to input the pain level on the device if the rest of the 5 vitals are already collected automatically by the patient monitor.

The patient monitor described above has a number of useful properties, including: (1) being configurable to receive six vital signs, via both manual input and sensor input; (2) adjusting the performance of the device based on several different variables such as user command and/or received patient data; (3) adjusting the patient data inputs, patient data parameters, and patient relevant output based on patient-specific algorithms and/or instructions provided by a doctor or health care provider; (4) establishing a connection with a remote server through a patient monitoring computer that is configured as a node in a wireless mesh network; and (5) working with a remote server, via a wireless mesh network, enabling the patient monitor to process these inputs to generate one or more alerts.

### EXAMPLE 2

### Patient Management and Monitoring System

A dynamically reconfigurable wearable patient monitor may be used as part of a patient management and monitoring system.

In one example, a dynamically reconfigurable wearable patient monitor may be referred to as a personal mobile physiologic assessment and safety assurance device (or “PMP”). As described above for the general dynamically reconfigurable patient monitor and the patient monitor example, this device may be worn by a patient that and used to monitor physiological data, to control therapy delivery, and to acquire manual assessments, for both patient monitoring and for diagnostic purposes.

In some embodiments, the wearable patient monitors described herein may be used as part of a system that includes additional components. As described in greater detail below, many of these system components may be integrated into the patient monitor, which may toggle between their functions, or perform their different functions simultaneously. In some embodiments, the systems include separate components that perform these functions (e.g., client, gateway, and router functions). In some embodiments, the system may include a plurality of multi-modal patient monitors in which one or more of the patient monitors performs the different functions.

For example, a system for monitoring a plurality of patients may include patient monitors (including reconfigurable patient monitors) and one or more servers. A wearable patient monitor may be used with a globally hosted server that retrieves patient data from a variety of sources, including a dynamically reconfigurable patient monitor. The server(s) can be managed by machine and/or by human moderators. Although a server may be remotely situated from the patient(s), an alert detected from collected patient data (either at the level of the server or at the level of the wearable patient monitor) may be transmitted to a care providers anywhere, in real time or at a pre-designated time. The modality of an alert transmitted to the care providers may be a variety of mediums, including email, fax, pager, cell phone, web page.

A systems for monitoring a plurality of patients may also include a client for interacting with (e.g., sending information to/receiving information from) one or more wearable patient monitors. A client may be a portable computer, PDA, notebook, laptop, or the like, in which client instructions (e.g., client software) is running. A client may also be referred to as a patient monitoring console (“PMC”) that can retrieve patient data, such as vital signs, from one or more wearable patient monitor, monitor this data, and transmit instructions and/or additional patient data to the wearable patient monitor. The client may also have wireless capability, for communicating with the wearable patient monitor(s) and/or a server or servers. The client may be set up as a network transceiver node that is plugged into any device including a processor (e.g. a computer, PDA, etc.). The node may be embodied in a USB key, and may include all software necessary to run client functions, configuring the device and its processor to include a patient monitoring console and coordinating communications with wearable patient monitoring devices in the vicinity, and the transfer of data to a server or servers. In some embodiments, the PMC may be identical in hardware to the PMP, but may possess a different set of software applications that allow it to function as a client.

In some embodiments, the system also includes one or more networked safety device controllers (“NSDCs”). An NSDC may be a controller to interact with a variety of devices, including medical devices such as infusion pumps and facility devices such as lighting. As mentioned above, one or more NSDCs may be integrated as part of a dynamically reconfigurable patient monitor, or the patient monitor may be configured to interface (wired or wirelessly) with one or more NSDC. An NSDC typically receives instructions either from the wearable patient monitor or relayed by the patient monitor to control a medical treatment device attached to the NSDC. For example, an NSDC may be networked to or through the dynamically reconfigurable patient monitor and thus in communication with another device in the body area network, or another device controlled by the server. The NSDC may therefore responsively control the treatment device as a function of the patient's physiologic data.

A device spigot (“DS”) is another component that may be included as part of a patient monitoring system. A DS may be a spigot connection that allows serial I/O to and from the device to be directed onto the mesh network. As mentioned, the wearable patient monitor may also include one or more integrated device spigots. A DS may be a multi-protocol device that can be remotely configured by the server to transfer data on a variety of networks, including the IEEE 802.15.4 mesh network. The DS typically has a unique identification key, which allows a server to identify the DS and the addressing scheme is directed to the DS's unique identifier. As was noted briefly above, the above devices (PMP, PMC, NSDC, DS) are different embodiments of patient information communication devices, and may share elements of the same basic hardware.

The devices and systems described herein may be used in any appropriate setting, including a hospital, hospice, home care, or in the field (e.g., in an emergency response situation). For example, in a hospital setting nursing practice typically requires periodically document patient vital signs on written charts. Oversight bodies such as the Joint Commission on Accreditation of Healthcare Organizations (The Joint Commission) require that Nurses periodically document patient's vital signs, anywhere from 15 minutes to 4 hours, onto written charts. This task is referred to as “rounding on patients,” as nurses visit patients under their care to take patient vital signs. This is a time-consuming and labor-intensive process, and can easily take up 30-50% of nurse's time. Recent clinical practices in acute care have included new methods (such as EWS, or early warning score, MEWS, or modified early warning score) for recognizing patient deterioration based on an analysis of the patient vital signs, but these methods require the regular and accurate measurement and documentation of vital signs. Nurses typically record vital signs on clipboards of notes, and, periodically, the primary physician reviews the notes during a diagnosis. The process of vital sign documentation has become a ritualized process, and is rarely driven by evidence or patient need. Written charts cannot effectively monitor patient deteriorations, and do not give nurses information to make decisions. The wearable patient monitors described herein may therefore provide advantages by subsuming many of these functions.

The wearable patient monitors and client components described herein may allow electronic entry of data at the point of care, allowing nurses or other caregivers to input their patient assessments, as well receiving and/or processing (storing, analyzing and/or transmitting) patient data such a patient vital signs and patient treatment devices.

Any of the wearable patient monitors described herein may also include one or more sensors. In some embodiments a system for monitoring a patient including a wearable patient monitor (such as a dynamically reconfigurable monitor or a multi-modal monitor) may also include one or more sensors or other data inputs. Examples of sensors that may be used with any of the patient monitors described herein include position sensors, environmental sensors, and vital sign sensors. Position sensors include: 3D accelerometer used to detect the mechanism and severity of injury or impact (e.g. for battlefield soldiers, for ski/snowboarders), 3D accelerometer monitoring the motion activities (e.g., monitoring the elderly patients), 3D accelerometer tracking, at a coarse grain, 3D accelerometer pattern detection and learning, for monitoring daily living activities, 3D accelerometer rehabilitation monitoring (e.g., for patients who are in a rehab center and need to be monitored on their position/activity characteristics), gyroscope-based dead-reckoning of the on-board GPS, ultrasound (e.g., MEMS ultrasound sensors to detect chest wall activity). Location sensors include: GPS monitoring of patient location (e.g., as a location tracking tool that may be particularly useful for Alzheimer's patients, children, or criminals on probation/parole), GPS monitoring of patient's altitude and location (which can be used to adjust the sensing behavior of other sensors), indoor location tracking technologies (e.g., 802.11 triangulation, active and passive RFID and UWB). Environmental sensors may include: temperature sensor detects hazardous temperature levels, toxic agent sensors, carbon monoxide sensors, ambient light sensor, sound sensors (e.g., to detect bodily sounds as well as external events). Vital Sign Sensors may include: heart rate, SpO₂, plethysmogram (e.g., using pulse oximeter), ECG (e.g., heart electrical activity), EEG (e.g., brain electrical activity), blood pressure sensors (e.g., systolic, diastolic, pulse), body temperature sensors (e.g., non-contact and/or contact sensors), CO₂ capnography, respiration rate sensors, non-invasive glucose sensors, galvanic skin response (skin resistance) sensors (e.g., to determine if body goes in shock, or sweats excessively). Vital signs monitored with an appropriate input may include: urine output, level of consciousness (alert, pain, voice, unconscious, etc.), pain level, cardiac contractility (e.g., based on leg raise exercise), core temperature (e.g., oral, rectal, etc.).

Any of the sensors described above may provide patient data, and therefore provide patient data inputs corresponding to the sensor (e.g., position data, vital sign data, etc.). Other patient data inputs include healthcare provider data input, patient data input, and input from treatment or monitoring devices. Examples of healthcare provider data input includes data that is entered manually, including data based on subjective diagnosis, (e.g., Pain Level based on a patient's self-assessment of the amount of pain the patient is in, level of consciousness based on the healthcare provider's assessment or based upon the patient's response, etc.), healthcare provider authentication data (e.g., RFID, fingerprint, smart card, username/password, etc.). Data may be entered in any appropriate manner, including: smart input card (e.g., a deck of instruction cards that is carried by the care provider, which may be inserted into the patient monitor to be recognized on a command or input from the card, such as entry for an alertness category based on inserting one of 4 cards that designates alerts), manual entry (e.g., forms entry), touch screen or button interface, free text entry, handwriting recognition, voice recognition entry (e.g., speech to text), smart card, or smart input card entry (e.g., with pre-defined set of input data). As mentioned, medication/medical procedures specific to the patient may also be entered, as well as assessments/notes. In some embodiments, patients may themselves be able to input data. For example, the patient monitor may prompt the patient to answer one or more questions and receive their answers. For example, “do you feel okay today?”, “have you exercised?”, “do you feel hard of breathing?”, and “does anything feel odd?”. The device may also be configured to permit the patient to input special conditions (e.g., “currently going to run a marathon.”).

The devices described herein may also receive input from one or more treatment devices, and a patient data input may include data input from a treatment device. Examples of treatment devices includes infusion pumps, inotrope therapy devices, IV-PCA (e.g., pain medication) devices, arterial-line blood gas and blood pressure sensor, invasive glucose sensor, anemia detector, or other medical devices placed on the patient, including other monitoring devices. For example, the wearable, dynamically reconfigurable patient monitor may receive information from an additional externally powered/controlled monitoring device. In some embodiments, the wearable patient monitor includes one or more ports (e.g., USB ports) that can be connected to a device such as a monitoring device or an expansion dock to which additional device can be attached.

A dynamically reconfigurable patient monitor may also receive input (including patient data input) from a client or server in communication with the patient monitor. In addition, monitor configuration instructions may be received by a server or client. Examples of Server-based inputs include: user interface display parameters, patient information (e.g., name, allergies, medical history, treatment course, etc.), patient classification (e.g., triage level), care provider input variables, patient input variables, monitoring algorithms, alert parameters for one or more patient data inputs, data input parameters (including parameters to adjust sensitivity of monitoring, sensors sampling rate, transmission rate, transmitted variables), flags to activate/deactivate monitoring algorithms or branches of monitoring algorithms, set the set of patient data inputs to be monitored (including, e.g., a list of external devices such as treatment devices, monitoring devices, etc.), and device diagnostics (e.g., battery check, sensor check(s), performance check, etc.).

As mentioned above, the patient monitoring devices described herein may also include one or more outputs for presenting patient status information (including patient data). In addition to patient data, the patient monitoring devices may also output instructions, information or data that is not necessary patient data. For example, the patient monitor may include a unique device ID or patient ID that is programmed into the device (e.g., into a flash memory). A device ID or patient ID may be either programmed at the factory, programmed over the wireless network, or programmed with the device after it is installed at the customer site. The patient monitoring device may also include network association data, including broadcast identification and handshaking information to initiate connection with server and external devices. Control data (such as control setting for any of the treatment devices described herein) may also be manipulated and presented by the patient monitoring device. For example, control setting for a treatment device may be used to activate, deactivate and control dosage of a treatment device. For example, the device may include or connect to a lighting control (for controlling illumination around the patient), nurse call system controls, etc.

The devices described herein may also provide information to the server or servers, in addition to the patient status and patient data. A patient monitoring device may provide self-calibration and self-check information. For example, at power on when requested by a server, a patient monitor may execute self-calibration routes to dynamically adjust its settings, and may provide notice to the server that it has calibrated, as well as any calibration information. In some embodiments, the devices may send configuration information (e.g., monitor configuration instructions) back to the server or client, including a confirmation that the configuration has or has not been achieved. Any of the devices described herein may also include power management information (e.g., battery charge, etc.), monitor status, monitor errors, etc. for transmission to a server or client. A dynamically reconfigurable patient monitor may reconfigure its performance parameters, including: monitoring parameters, specificity and sensitivity of alert detection parameters, types of alert detections used, data collection parameters, sampling rate of sensors, accuracy (floating point integer size) of sensed data, types of sensed data, wireless parameters, frequency of wireless transmission, storage parameters, frequency of storage, size of storage, types of storage, etc. These performance parameters may be adjusted by modifying or providing a new set of monitor configuration instructions, indicating which patient data inputs to monitor (e.g., what sensor, what treatment devices, etc.), how these patient data inputs should be monitored (e.g., parameters for monitoring them), control information (e.g., instructions for controlling treatment devices or sensors), algorithm information (including any instructions for analyzing and/or presenting patient data), alert or analysis parameters, etc.

The monitor configuration instructions may be generated de novo for each patient, or they may be selected from a menu of predetermined instructions. In some embodiments the instructions are task-specific. For example, the device can be used to assist in a variety of tasks, including heart diagnostic, heart monitoring, respiratory monitoring, etc. A device may adjust its parameters based upon the healthcare provider's goal. In some embodiments, the instructions are disease-specific (e.g., instructions are tailored to patient diagnosis such as sepsis, pneumonia, respiratory disorders, heart failure, etc). In some embodiments, the instructions are based specifically on patient needs, including patient's prescribed medications, pre-existing conditions, physician's specified algorithms and parameters, etc.

Information may be presented by the device, and may be tailored by parameters provided in the monitor configuration instructions. For example, patient status or data may be presented on a display (e.g., on the patient monitor or separate user console), and may be adjusted to a specific user interface (UI) depending on who is reading the UI, so that it can display appropriate information. For example, if a patient is reviewing UI, it may display a Question and Answer screens for completion by the patient. If a nurse is reading UI, it may displays only information necessary to the nurse, and allow the nurse to input patient assessment data, and generate real-time alerts for any assessments that show dangerous levels. As mentioned above, an RFID or fingerprint reader may be included to detect the reader, authenticate the reader, and display appropriate information.

The patient monitoring devices can also communicate with a mesh network. Mesh network messages can be single hop or multi-hop, reliable or unreliable retransmission. Single hop reliable messaging is frequently used to communicate to therapeutic devices nearby the patient, because restricting a transmission to single hop may limit the message to only devices nearby the patient, as a safety measure. Multi-hop messages are frequently used to communicate with monitoring consoles (e.g., situated at the nursing station) and servers that may be far away from the patient. Reliable messaging (retransmission of data until an acknowledgement is received) is used for high priority vital sign data such as when a patient is in an alert state or when the information is used to provide critical therapeutic device decision support.

### EXAMPLE 3

### Applications of Dynamically Reconfigurable Patient Monitors and Systems

The dynamically reconfigurable patient monitors described herein may be used in a variety of diverse patient care scenarios, a few of which are illustrated below. In one illustration, a dynamically reconfigurable patient monitor may be used with a congestive heart failure patient who goes to the emergency room due to a fever. In admitting, a wearable patient monitor is given to the patient, and that particular monitor is associated with the patient (e.g., at the level of the server and/or at the level of the patient monitor). The wearable monitor may be initially set in an “admitting” mode, in which the monitor is configured to receive patient identification data (e.g., patient-specific information such as name, allergies, initial complaint, overall medical condition, etc.), and to monitor all or a subset of patient vitals (e.g., heart rate, temperature, pain level, etc.). After examination by a healthcare provider, the monitor may be reconfigured to more specifically monitor the patient. For example, if the patient's doctor suspects that the patient may have sepsis, she may set the patient's wearable monitor into a “sepsis monitoring” mode, in which vital signs relevant to determining sepsis are specifically monitored, and may be processed by an algorithm that uses these vital signs to provide an estimate of patient condition with respect to sepsis. For example, the monitor may be set to receive patient data for all or a subset of: blood pressure, body temperature, respiratory rate, white blood count, heart rate, etc. Thus, the caregiver may select monitor configuration instructions including sepsis monitoring instructions. These instructions may indicate a set of patient data inputs to be monitored, including blood pressure, temperature, respiratory rate, WBC, and heart rate. The monitor configuration instructions may also indicate the patient data input parameters for each of these patient data inputs, such as parameters instructing the monitor to confirm (or request) connection to the appropriate data input device, and parameters controlling the sample rate, gain, etc., for each patient data input. In addition, the instructions may include patient data alert parameters specific for sepsis. In one embodiment, these parameters include instructions or algorithms that may be used by the monitor's processor to estimate risk of sepsis. For example, Rivers et al. (“Early Goal Directed Therapy in the Treatment of Severe Sepsis and Septic Shock, The New England Journal of Medicine, vol. 345, no 19, Nov. 8, 2001) indicated a treatment algorithm to identify patients at risk for sepsis by the SIRS criteria and by BP. Thus, if a patient meets the SIRS criteria (temperature greater than or equal to 38 C or less than 36 C, heart rate greater than 90 bpm, respiratory greater than 20 bpm, and WBC greater than 12,000 or less than 4,000) and the systolic blood pressure is less than 90 or lactate is greater than 4 mmol/liter, the patient is at risk for sepsis and should receive the time sensitive, goal directed therapy. In this example, although the patient monitor is placed in a sepsis-monitoring mode, it may also concurrently monitor other vital signs or patient data, and also process this patient data to detect other indicators of patient condition. This may be achieved by expanding the monitor configuration instructions, for example. Furthermore, these instructions may be modified on-the-fly, either by the patient's caregiver (e.g., doctor) or by the patient's condition.

In another illustration, patient ‘Norman’ arrives at the emergency room, complaining of a fever. Norman goes to the registration desk to check in, and the registration nurse puts a dynamically reconfigurable patient monitor on him. From her computer console, the nurse inputs the patient name, address, chief complaint (“fever”), pain level. Her computer console communicates with Norman's patient monitoring, and set it into a preliminary monitoring mode, in which it automatically monitors the real-time vital signs, and transmits them to the console (e.g., client console) viewed by the nurse. This client console receives this information from Norman's wearable patient monitor. In this initial mode, Norman's monitor may receive and transmit to the Nurse's computer console 5 of Norman's vital signs. Alerts may be set at either (or both) the Nurse's client console or at the wearable monitor (e.g., as part of the monitor configuration instructions) to indicate if any of Norman's vital signs indicate a potential problem.

In some embodiments, the wearable patient monitor contains physiologic sensors. For example, the wearable patient monitor may include sensors that automatically record 5 of the patient's 6 vital signs. The 6th vital sign, the “pain” score, may require the nurse to ask the patient (e.g., Norman) to describe how much pain he is in, on a scale of 0 to 10. This information may also be entered into the wearable patient monitor and recorded or transmitted. Alternatively, the wearable patient monitor can prompt Norman to input this information directly himself. For example, Norman states that his pain level is about 6. The pain score can be entered manually, either into a client computer console or directly into the wearable patient monitor.

In some embodiments, the wearable patient monitor does not include all of the physiologic sensors collecting the necessary patient data input that the device is dynamically configured to receive. For example, a thermometer may not be included as part of the monitor. In this case, the monitor may detect a connection to the physiologic sensor (e.g., thermometer) and, if no device is connected or within communication range (via wireless connection) to the monitor, it may send a prompt to request connection. This prompt may be presented at the patient monitor (e.g., via. LED or message), and/or it may be transmitted to the caregiver (by way of the client or server). For example, the wearable patient monitor may send an alert to the Nurse, requesting connection to the appropriate monitoring device.

Continuing with the illustration involving Norman, the nurse may triage Norman at triage level 3 at the registration desk, and this information may be communicated to his wearable monitor. Norman is triaged to a triage level 3 patient based on the nurse's initial estimate of his condition. In this example, the hospital has 5 priorities, levels 1 to 5, indicating patient condition in decreasing order of illness severity. When priority 3 is set, the wearable patient monitor may display a priority 3 indicator on the display (at the nurse's client console, and/or at the wearable monitor). The monitoring server may monitor the priority 3 status, and any alerts may be adjusted to an appropriate acuity for a priority 3 patient.

Typically, every four hours a nurse in a hospital ward may round on her patients to record patients' vital signs. Traditionally, this has involved a nurse carrying around a clipboard for each patient. The clipboard may include the patient's charts, and the 4 hour recordings of their vital signs (6 vital signs are recorded every four hours, as required by The Joint Commission). The nurse may push a around a cart holding one or more vital sign monitoring machines. For example, the nurse may take the patient's pulse and heart rate with a pulseoximeter, then takes the temperature using an IR temperature probe, and inflates a blood pressure cuff. This can easily take 5 minutes, after which the nurse records all this information on a piece of paper. Finally, the nurse wipes down the machine(s) with an antiseptic wipe (also a required step by The Joint Commission) and moves onto the next patient.

Using the wearable patient monitors described herein, the nurse can simply walk to each patient in the ward, without carrying necessarily having to carry any devices or clipboards. The patient is already wearing a dynamically reconfigurable monitor, so the nurse simply reviews the wearable monitor, and can attend to any prompts or alerts provided by the monitor (e.g., to connect the patient to a therapeutic or measurement device, to ask the patient for a pain level, etc.), and can enter any information into the device that is not already recorded. For example, if the patient is being monitored for the Joint Commission-required vital signs, the nurse may only have to input the pain level on the device, and the remaining 5 vitals are already received (and transmitted or recorded) by the patient's wearable monitor.

The patient data received by the monitor may be time stamped. For example, the monitor may include a clock for time stamping the data, or the server may timestamps the information that is received, thus recording the time that the nurse has monitored the patient. Four hours later, the nurse could receive a page, initiated by the server or by the monitor, to remind the nurse that it's time to take Norman's vital sign readings. This usage scenario may allow the wearable patient monitor to be integrated into the day-to-day workflow of the nursing practice without introducing any changes to the workflow. In some embodiments, the nurse may not need to round on patients, as patients may be monitored from a central service location.

In some embodiments, patient monitoring may be based on disease-specific patient monitor configuration instructions. For example, if there are 20 patients in an intensive care unit, whose conditions and diagnoses all vary, and their doctor must keep track of each or their conditions, specific to the disease of the patient. The clinician may use a system including wearable, dynamically reconfigurable patient monitors for each patient to select diagnosis criteria for each patient. For example, individual patient monitors may be set to monitor infection criteria, SIRS criteria, acute organic dysfunction criteria, burn criteria, etc. Based on the patient's specific disorder, instructions may be sent to each patient monitor to set the configuration instructions. For example, client (or server) software may help the physician select the appropriate configuration instructions, and may translate treatment criteria into monitoring algorithm parameters, and transmits these monitoring parameters to the dynamically reconfigurable monitor. The monitor receives the instructions, including parameters for monitoring and processing the patient data, and reconfigures its performance parameters and user interface display so it would only prompt a caregiver to enter the inputs as required by the diagnosis criteria. For example, a nurse may approach each patient and review the patient's wearable monitor display (or information sent to her client monitor), to review the vital signs that needs to be recorded based on the prescribed criteria for that patient's condition. The wearable patient monitor may automatically display diagnosis criteria as well as patient data, so that the basis of the configuration instructions is clear.

In some embodiments, the patient monitoring is based on patient-specific assessment. For example, ‘Marge’ is a victim of an automobile accident. Marge gets into the ambulance, where she is given a dynamically reconfigurable patient monitor to wear. Her monitor is set to “diagnostics” mode, and adjusts its data acquisition rate to collect sufficient data for diagnosis based on the set of patient data inputs to be monitored (from the configuration instructions for the ‘diagnostics mode’) and the patient data input parameters for each of the patient data inputs (e.g., including parameters controlling the sampling rate, transmission rate, transmission data types, sampled data types). The monitor may receive signal from all 12-leads of the ECG and transmits this data in real-time, with no delay, to the receiving server. Transmission to the server is via a global network, such as cellular, satellite, or WiMax. When the device is in diagnostic mode, it may operate in a “high acuity” sensing state, which allows the detection of conditions such as atrial arrhythmias, ventricular arrhythmias, ST segment elevation/depressions, and other artifacts of her ECG. Marge is about to be sent to a Level 1 trauma center. The trauma surgeon at the receiving center carefully observes Marge's 12-lead ECG rhythms while Marge is on the ambulance. He prepares for surgery and calls the necessary medication and surgical supplies. Marge's ambulance arrives, and she is wheeled into the hospital. She is headed toward the trauma operating room (OR). Without the dynamically reconfigurable wearable patient monitor, her first 5 minutes in the OR might be spent on strapping on vital sign sensors (ECG, A-line blood pressure, etc.) and adjusting the monitoring machines to calibrate. However, because she was already outfitted with a reconfigurable patient monitor that can communicate with the server accessed by the hospital during her ambulance ride into the hospital, she does not experience this delay. Her monitor includes a 12-lead ECG, records her vitals, and all the necessary vital signs, so that any surgery can start as soon as she is wheeled into the OR.

After surgery, Marge goes to the post anesthesia care unit (PACU), where she will be monitored for recovery. Marge's monitor may then be reconfigured and set to PACU monitoring mode. She is put on an opioid (pain medication) through a device called IV-PCA. The opioid is injected into her IV through a PCA opioid eluding device. Marge can activate each dosage of drug with a button. The nurse may augment Marge's IV-PCA with a safety control device (e.g., PANDC), which communicates with Marge's wearable monitor. The wearable monitor sends control signals (and/or vital sign data) to the PANDC, so that as long as Marge's vital signs are within a pre-specified threshold, the PANDC remains passive. If Marge's vital signs exhibit a respiratory depression (often an indicator of the onset of opioid overdoes), the PANDC can alert the clinician, and discontinue her ability to dose herself. In some embodiments, the functions of the PANDC may be performed by the wearable monitor. It can discontinue dosage by shutting off power to PCA device, deactivating her button, or occluding the IV line.

After the PACU, Marge is sent to a burn step-down unit, where she will complete her recovery. She is schedule to stay in the step-down unit for 4 days. While in the step-down unit, Marge's wearable monitoring device is set (e.g., by a nurse) to “step-down unit monitoring mode”.

In some embodiments, patient monitoring may be based on a prescription-driven basis. For example, the dynamically reconfigurable monitoring device may be configured based on a monitor configuration that is based on physician-determined diagnosis and course of treatment. For example, systemic inflammatory response syndrome (SIRS) can be a limited condition, or it can progress to septic shock. In the continuum between SIRS and septic shock, circulatory abnormalities result in an imbalance between oxygen supply and demand. Clinical end-points have been defined by Rivers et al. to improve the balance of oxygen supply and demand and to reduce the mortality of septic shock (Rivers et al., “Early Goal Directed Therapy in the Treatment of Severe Sepsis and Septic Shock, The New England Journal of Medicine, vol. 345, no 19, Nov. 8, 2001).

In order to apply the treatment algorithm outlined by Rivers, patients need to be identified as at-risk for sepsis by the SIRS criteria and by blood pressure (BP). For example, a patient may be at risk for sepsis and should receive the time sensitive, goal-directed therapy as indicated by Rivers et al if the patient meets the SIRS criteria (temperature greater than or equal to 38° C. or less than 36° C., heart rate greater than 90 bpm, respiratory greater than 20 bpm, and WBC greater than 12,000 or less than 4,000) and systolic blood pressure less than 90 or lactate greater than 4 mmol/liter.

As an example, ‘Jane Doe’, a 40 year old female with a congenital kidney disease, is on the transplant list. Ms. Doe has had recent infections and has difficult venous access. Her PIC line was not removed with her most recent infection in order to keep access for dialysis. Her clinicians believe the infection has cleared. However, her PIC line could be contributing to the infection, allowing the infection to return with improved resistance to antibiotics. The repeat infection can easily seed the blood and send Ms. Doe on the path to sepsis. Ms. Doe's physician can program her dynamically configurable patient monitor to send her real-time SIRS score to a client or server (e.g., a nurse monitoring station and/or to a physician's PDA or pager). If Ms. Doe's SIRS score becomes positive and her systolic blood pressure drops below 90, her physician and/or nurse will receive an alert immediately. They can then take action during the critical “golden hours” during which the Rivers goal-directed treatment provides maximal benefit.

A physician interface may be used to enable the physician to select the monitor configuration instructions to match the acuity of the patient to the necessary clinical algorithms and vital sign thresholds for which alerts are triggered. As part of the admission orders, the physician may include a list alerts, or scenarios under which the nurse should call the physician: call if HR is greater than 100 or less than 60, if systolic BP is greater than 160, etc. A physician can electronically select the algorithm or vital sign thresholds for which an alert is triggered (e.g., from a menu of monitor configuration instructions including patient data alert parameters). For example, Ms. Doe's physician can choose for the nursing alert to be triggered if the SIRS score is positive and can select a physician pager alert to be triggered if the SIRS score is positive and the systolic BP is less than 90. This may create less work for the nursing staff, and may decrease the physician's dependence on the nurse's ability to gather all the data in a timely manner.

### EXAMPLE 4

### Embodiments of Dynamically Reconfigurable Patient Monitors and Systems

In addition to the features described above, a dynamically reconfigurable patient monitor may act as an “electronic clipboard” that allows manual data input. The patient monitor may automatically processes the inputted data along with data received from any of the additional patient data (e.g., data received from external sources such as sensors or other devices), and can also pass the data on to a client or server. In addition, alerts (e.g., based on patient data alert parameters) may be triggered based on the user data. Prompts, data and alerts may be displayed on an onboard display. For example, the device may prompt the user to input additional information, instruct the user on how to perform certain tasks, or display alerts with the patient's data.

In some embodiments, the wearable patient monitor allows the manual, automated sensor, and electronic device input of: patient vital signs, patient assessments, patient medical history/records/allergies, intake and output, and medication times. The devices (or systems including the devices) allow input of periodic and continuous inputs, can processes the combination of the two input feeds, and can then generate continuous feedback to the user via onboard display based on the two forms of inputs.

As described in more detail below, the devices may be part of a network, including a mesh network, and may therefore also receive and pass on data or information from other patient monitors, and may display information from or about other patient monitors.

In some embodiments, the wearable patient monitor may include a bar code scanner for medication compliance, identification of care provider, blood transfusion verification, lab specimen identification, external input devices, or other use. An integrated barcode scanner may prevent the need for a separate barcode scanner, and could enhances patient safety by making this barcode scanner anywhere the patient is.

In some embodiments, the wearable patient monitor may receive patient data input from a healthcare provider (e.g., nurse, doctor, patient, etc.), including textual, voice, or image data. The patient data monitor may dynamically create fields for the patient data input to record information directly onto the device itself. For example, monitors may generate fields on the fly to support structured clinical documentation workflow, and reduce insufficient input. Examples of manual input fields include: “annotate data” (so the caregiver can place an annotation into the continuous sensor data before it goes into the patient record), “assessments” (providing qualitative information such as how the patient looks and feels, level of consciousness, pain level, respiratory effort, capillary refill, etc.), “triage information,” “chief complaints,” “allergies,” “medications,” etc.

In some embodiments the monitor displays feedback to the patient, for patient education. For example, it may provide directions on how to connect or apply a sensor such as a pulse oximeter clip back on, or reassuring information such as “your ambulance is on its way,” or the like.

Any of the dynamically reconfigurable monitoring devices described herein may also be dynamically reconfigured based on the patient data received. This may be controlled by the on-board processor, or by comments from a client or server that received the patient data. For example, a patient monitor may self-configures its operation to collect more patient data, to collect patient data from an additional patient data input (or remove an input), to display instructions to the user, etc. In one embodiment, if the patient's heart rate (HR) is suddenly increased, the monitor may check the patient's temperature. Thus, the patient monitor may automatically turn on the temperature sensor to check the temperature, and if the temperature sensor is not available on the device, it could display a message to the healthcare provider (e.g., through the on-board LCD or wirelessly to a client/server) to manually input the temperature reading or connect a thermometer.

In one illustration, a nurse comes to the patient to collect vital signs. She logs into the patient's wearable patient monitor, and decides to enter a new dosage of medication Y into the dialysis machine. She scans information (e.g., the name of the medication Y) about this into the patient monitor, and the monitor tells her that the patient's blood pressure must be checked before she gives the dosage. She then checks the patient's blood pressure by connecting the patient monitor into the a blood pressure cuff (e.g., via a serial output cable). The patient monitor then displays the new blood pressure and also transmits this information over the wireless network. The monitor can therefore confirm that the BP is within range and can indicate “OK to administer medication Y.” The nurse then administers the medication, and the monitor can prompt her to “check temperature.” The monitor may then prompt her to “check Respiration Rate,” and may indicate when all of the recommended or requested vital sign checks have been completed (e.g., “vital signs completed. Vital signs within normal limits for this patient.”). The monitor may also provide instruction to the nurse on where to go next, because it was able to prioritize the patients in her list (e.g., “Proceed to patient Bob Jones in room 22.”).

In another illustration, a medic approaches a patient at a mass casualty disaster. The medic places the dynamically reconfigurable and wearable patient monitor on the patient. The monitor may be set in “emergency response” mode, and set to detect the patient's vital signs and automatically makes a projected diagnosis on the patient. If the vital signs are normal, the monitor may display “Normal Condition.” Ten minutes later, if the patient's vitals start to drop, the medic (who may be working on another patient), may be prompted by a message that the first patient is having problems. This message may be received on the medic's client device (e.g., PDA) or on the other patient's wearable patient monitor that is part of the same network. The medic can then return to the first, and the wearable monitor can prompt the medic to record the “level of consciousness”. In a mass casualty incident, the monitor may not prompt the medic to enter any more detail than what is necessary to monitor the patient's health. During a medical emergency, when time is of essence, the medic is not required to spend any more time entering information that may be unnecessary for monitoring the patient at hand. So the device may intelligently monitor the patient to detect deterioration in the patient's condition, and respond properly when deteriorations occur. In one instance, the monitor may respond by querying the medic to collect more data, only so the monitoring may be refined to improve patient treatment.

In general, the devices may help assist in treating and diagnosing patients based on recorded patient data. For example, the system may use newly inputted patient information to refine its monitoring of the patient, as indicated above. In some embodiments, the monitoring system does a pattern match of the patient to an expected deteriorating condition.

### Methods and Devices for Managing a Plurality of Patient Monitors

A plurality of patients may be efficiently monitored with any of the wearable patient monitors described herein by applying triage rankings. In some embodiments of the devices described herein, the monitors and/or other parts of the monitoring system generate and apply triage rankings to control the flow of information between different parts of the system, such as between individual wearable patient monitors, clients and server(s). The methods applying triage ranks described herein are particularly useful when the wearable patient monitors are part of a wireless (or partially wireless) mesh network, in which information is passed between individual nodes (e.g., each wearable monitor may be a node) and received by a client device and/or a server.

A mesh network is a communications network that permits two or more paths for information to flow between any node. In a mesh network, data can be routed between nodes so that continuous connections and reconfiguration around broken paths can be made by “hopping” from node to node until the destination is reached. Mesh networks may be referred to as self-healing, because the network can still operate even when a node breaks down or a connection goes bad. As a result, a very reliable network is formed. This concept is applicable to wireless networks, wired networks, and software interaction.

In systems having only a few nodes (e.g., a few patient monitors), traffic on the network is not likely to be a problem. However, as the number of monitors increases, or the amount of data collected by individual monitors increases (e.g., depending on the configuration of the monitor), transmission of information from individual nodes to the server may become delayed. This is particularly true when ‘bottlenecks’ occur, in which all or most traffic to the server must pass through a single node. Delays in the passing information from patient monitors to the remote server(s) or the client monitor (e.g., nurse's monitor) are highly undesirable, because it may endanger patient safety. Prioritizing information passed on the network may organize network traffic and ensure safety and efficiency.

A triage or priority rank can be dynamically determined for each patient monitor, so that information from that patient monitor (and, in some cases, information sent to that patient monitor) is associated with that priority rank to prioritize transmission at each node in the network. The priority rank may be based on based on (1) the medical or patient content of the information transmitted and/or (2) the status of the patient, and/or (3) the patient's condition, and/or (4) a command from a healthcare provider. The priority rank is dynamic because it can be modified on the fly—as the patient's condition changes, or as other patient's are monitored. Thus, priority rank can be adjusted as information from each monitor is transmitted to the client(s) and/or server(s) in the system.

In most of the embodiments described herein, the priority rank is established for all information transmitted by (and in some cases to) an individual patient monitor. Although the rank may be changed, the priority rank typically refers to the rank of the monitor (and therefore the patient). However, in some embodiments the priority rank is determined for all or some individual messages sent by the patient monitor.

In general, the method for monitoring a plurality of patients using patient priority ranks involves determining a first priority rank for a patient monitor, coupling the priority rank with information transmitted from that patient monitor, and then transmitting the information and the coupled priority rank. These steps may be performed for each monitor in the system (thus, for each patient). Information transmitted by a patient monitor may be received and retransmitted by other nodes in the system, and the priority rank associated with that information can be compared with the priority rank of other information waiting to be passed by that node, and information having a higher priority ranking may be passed first. If a tie in priority rank occurs, the decision of which information to pass first may be determined by secondary parameters, such as a time indicator (time stamp) on the data (so that earlier information is transmitted first).

Priority rank may be determined by weighing different parameters relevant to each patient monitor. For example, priority rank may be determined in part by the monitoring mode of the patient monitor, for example, surgical monitoring versus recovery room monitoring. Priority rank may be determined in part by the status of the patient, such as the patient vitals, and particularly the triggering of any alerts. Less normal vitals may increase priority rank, for example. Priority rank may be determined in part by the patient's condition, such as patient diagnosis or prognosis. Patients having more life-threatening conditions, or more volatile conditions may be given higher priority ranks. Priority rank may also be determined in part by instructions or commands from a client or server, based on instructions from a healthcare provider such as a doctor or nurse. For example, the doctor may wish to increase the priority level of a patient she believes should be monitored more closely.

The ultimate priority rank may be based on a weighting of each of these parameters. In addition, the priority rank may be changed as monitoring continues and one or more of these parameters changes. Thus, the monitor may continuously or periodically adjust its priority rank. The actual weighting of the priority rank parameters may be changed as well (e.g., by reconfiguring the patient monitor). In some embodiment, the wearable patient monitors include priority rank setting logic to determine the priority rank for that monitor. Priority rank setting logic may be instructions (e.g., software, firmware, etc.) that may be executed on a processor or controller (e.g., the monitor controller and/or processor) of the device.

To apply the principles described above, each node in the wireless network (including relays, gateways, servers, clients, monitors, etc.) may apply priority comparison logic to determine the order of transmission of information based on the priority rank associated with that information. For example, each node in the network may have a controller or processor (such as a dedicated wireless controller and/or processor, or a general monitor controller and/or processor) configured to compare the priority ranking and determine the order of transmission (or retransmission). Information from each monitor (or from the client, server, or other component of the network) is routed through the nodes base on the priority ranking associated with the information (e.g., based on the priority rank of the node from which the information originated or is destined).

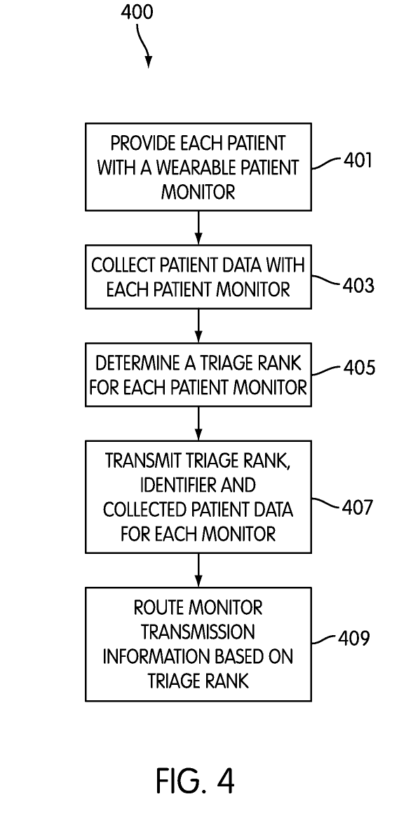

FIG. 4 is a flow diagram illustrating one embodiment of a method 400 of monitoring a plurality of patients using priority ranks. Method 400 begins by providing each patient with a wearable patient monitor. These monitors act as nodes in a network of patient monitors (as will be described below). Each patient monitor in this example includes a wireless transmitter/receiver (which may be separate elements or a single element) and a plurality of patient data collectors configured to receive patient data. Each patient monitor also has a unique identifier associated with it.

Method 400 continues with task 403, and patient data is collected with each patient monitor, as described above with respect to methods 200 and 300. Based on that collected data, a priority or triage rank for each patient monitor is determined in task 405. For example, the priority rank may be a numeric value within a predetermined range, such as between 1 and 5, where 1 is the highest rank, and 5 is the lowest. Thus, 1 may correspond to critical condition, and 5 may be stable, with intermediate ranks in between. As mentioned above, the patient monitor may determine a priority rank once (e.g., on activation of the monitor), or periodically (e.g., based on a predetermined schedule such as every half hour, etc.), when triggered (e.g., by a command from a server or client, or when an alert is tripped on the monitor), or continuously.

Patient data collected by the patient monitor may be stored and/or transmitted. As was described above, information to be transmitted may be prepared by associating it the unique patient monitor identifier. Patient data to be transmitted may also be associated with the priority rank. The patient data and the priority identifier (and monitor identifier) may then be transmitted, as shown in task 407. Patient data may be routed during transmission, as will be described below in more detail. When the wearable patient monitor is part of a wireless mesh network, the patient data can be received by another node, repeater or router, and forwarded on; at this step, data is forwarded based on the priority rank. When data is transmitted for the first time from a node, that same node may be receiving information (or may have received information) to along. Thus, in task 409, even data originating from this node is transmitted base on the priority rank associated with the data. Method 400 concludes after task 409, but may be executed again, either continuously or at intervals.

By applying priority ranks when transmitting data, the wearable patient monitors described herein may intelligently route patient data appropriately based on network congestion and priority level of data. For example, the wearable patient monitor may forward data from high priority patients (high priority ranking patients) before its own (lower priority level) patient data is forwarded. In addition, a wearable patient monitor may also streamline transmission of patient data during periods of network congestion by at least partially reconfiguring themselves based on network traffic conditions.

For example, the wearable patient monitors may reduce the traffic by reducing the amount of data sent. This may be based (in part) on the priority level for the monitor. For example, the wearable patient monitors may adjust the sampling rate of data (e.g., by lowering the resolution/sample rate of low-priority data). This may be accomplished by modifying the monitor configuration instructions (particularly the patient data input parameters), as mentioned above. Thus, the priority rank may be used to adjust the sample rate, particularly if some indicator of network traffic (e.g., sent to patient monitors by the server) indicate a high level of network traffic.

### Multimodal Patient Monitors, Systems and Methods of Use

In the description above, patient monitors according to embodiments of the invention are referred to as “reconfigurable” because the tasks that they perform, the data that they collect, the methods by which they report that data, and their user interfaces can be reconfigured to suit the patient and the context. Additionally, any of the wearable patient monitors described herein may be multimodal, in that a single monitor may perform multiple functions in a network. As the term is used here, multimodal patient monitor refers to a wearable patient monitor that is competent to act as one or more of: a patient monitor, a mesh network node, a gateway, a mesh network router, a repeater and a console. Of those terms, a node is a device that participates in a network; a gateway connects two networks, such as a wired network and a wireless network; a router determines the path that data should take between two nodes in a network; and a repeater retransmits data that has been received so as to improve the range or reliability of a network.

Generally, the multimodal patient monitors may be toggled between these different modes (e.g., from acting as a patient monitor and a client node). In some embodiments, the device may operate simultaneously in two or more of these modes. Because they are capable of multimodal activity, multimodal patient monitors may be used to form the backbone of a patient monitoring network, without requiring dedicated components.

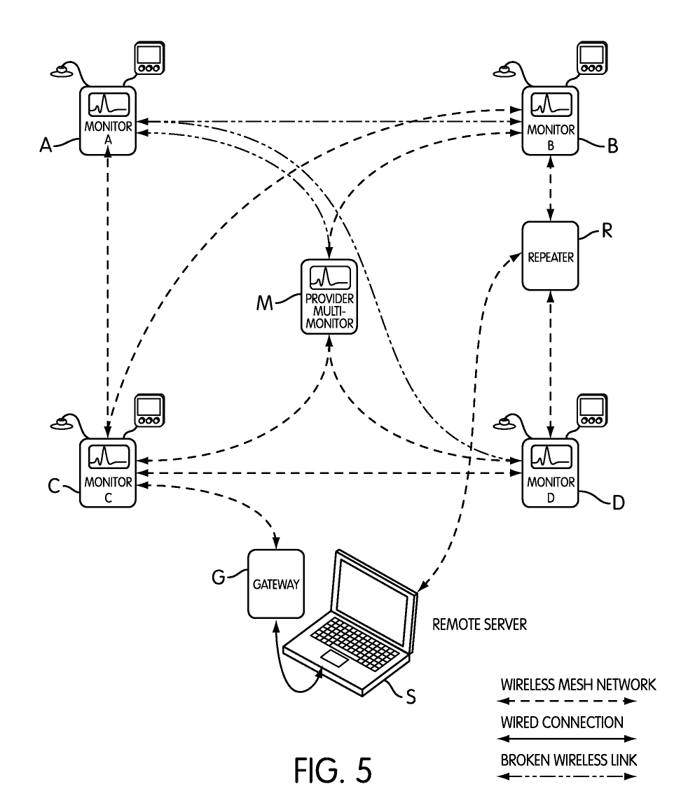

FIG. 5 is an illustration of a system using multimodal patient monitors. In FIG. 5, each of the nodes of the ad-hoc mesh network (e.g., patient monitor A, patient monitor B, patient monitor C, and patient monitor D) may be multimodal patient monitors that are set to act as mesh network nodes and patient monitors. In this mode, the patient monitors can monitor patients as described above (e.g., as dynamically reconfigurable patient monitors) and transmit patient data on to other nodes and eventually to a client (e.g., multimonitor M) or server S. Because patient monitors A-D are also nodes, they may relay information to and from other nodes, client(s), server(s), etc. In the network shown in FIG. 5, the Nurse patient monitor M is set to client mode. In client mode, the patient monitor can execute client logic (e.g., running client software) to monitor and interact with a plurality of patient monitors, such as patient monitors A-D. In the client mode, the patient monitor receives and displays patient data, and can also control all or a subset of these patient monitors, and also communicate with the server. For example, a nurse operating a multimodal patient monitor M set to client mode can enter observations or other patient data on patient's (e.g., by entering information from the client patient monitor and having the client patient monitor transmit this information to the patient's monitor or directly to the server). If a patient monitor (e.g., patient monitor) triggers an alert (e.g., if the patient's vital signs become unstable), this alert can be passed to the client to alert the nurse or other caregiver.

In FIG. 5, the patient monitor repeater node R is a multimodal patient monitor set to mesh network router mode. In this mode, the device operates as a router to pass along information to/from other nodes in the patient monitoring network (including client and server). The patient monitor repeater does not monitor a specific patient, but merely passes on information to and from other monitors.

Similarly, in FIG. 5, the patient monitor configured as a gateway G is a multimodal patient monitor that is set to ‘gateway’ mode. In this mode of operation, the device can connect the mesh network of patient monitor to one or more additional networks or one or more additional devices (e.g., medical devices), including connecting the mesh network to a server. For example, a multimodal patient monitor may act as a gateway between the mesh network and the internet or a dedicated intranet. In FIG. 5, the multimodal patient monitor includes a port or connector so that device can be connected to another processor (e.g., a computer or server S). For example, the port may be a USB port. In some embodiments, the gateway mode may connect the mesh network to devices that are not part of the mesh network (but not necessarily part of a second network). For example, the gateway mode may allow the connection of the multimodal monitor to a medical device that is a stand-alone device (e.g., an IV-PCA, etc.) and place that device on the patient monitor network. This allows the multimodal patient monitor to link to a medical device and act as a wireless gateway for the medical device.

As is indicated by the broken and solid arrows in FIG. 5, the connections between the patient monitors A-D, the monitor in client mode M, the repeater R, and the gateway G are wireless. The nature of a mesh network provides redundancy. For example, in FIG. 5, direct connections between some elements in the network cannot be established or are broken. For example, a connection between monitor A and the nurse client monitor M cannot be established; however, monitor A can communicate with the client monitor M by sending its data to monitor C, which will then relay it to the client monitor M.

The number of hops between monitors may be taken into account in prioritizing and processing data. For example, a client monitor M may, in some embodiments, accept data only from those patient monitors A-D that are one hop away, and may reject or not display the other data. That can be useful, for example, in triage situations where the medical professional who is performing triage may only be interested in seeing data from monitors A-D that are immediately proximate to him or her.

The patient monitors A-D and multimonitor M may or may not be equal in capabilities. In each case, the processor of monitors A-D and the multimonitor or client monitor M may determine which data should be displayed on the local device or on a remote device, which data should be processed on the local device versus a remote device, and which data should be stored on the local device, versus a remote device. Data from devices of lesser capability may be sent to devices of greater capability for storage, processing, or display. Thus, each device can act as a data source, a data forwarder, or a data sink (i.e., a data recipient). The data transmitted and received by the devices may be associated with a single patient, associated with multiple patients, or not associated with any particular patient.

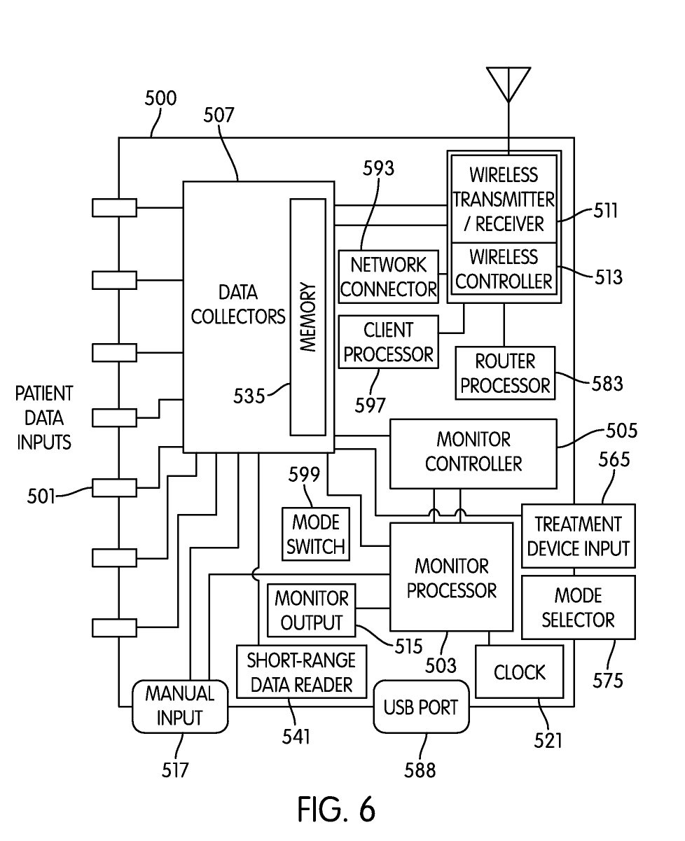

FIG. 6 schematically illustrates one embodiment of a multimodal patient monitor competent to act as a mesh network monitor node, a gateway, a router, and a client. As mentioned, a multimodal patient monitor may be toggled between these modes, so that the device acts as a patient monitor (and may be a wearable patient monitor that is dynamically reconfigurable, as described above). A multimodal patient monitor 500 may also include a mode switch 599 configured to switch the device between these modes. When toggled as a patient monitor to be worn by a patient, the device may operate as a mesh network monitor nodes (e.g., as a node in a mesh network), or it may act as a stand-alone monitor (not part of a mesh network). The device may also be switched into any of the other modes (e.g., gateway, router, client). In some embodiments, the multimodal device includes only a subset of these modes (e.g., monitor/client, monitor/gateway, monitor/router, monitor/client/router, monitor/client/gateway, monitor/router/gateway, etc.). Furthermore, multiple modes may be selected simultaneously, so that the same device acts as both a monitor and a client, as a monitor and a router, etc. For example, a nurse may temporarily use a patient's monitor to view other patient data (for other patient's wearing monitors) by temporarily switching the mode of the patient's monitor into client mode. The device may continue to monitor the patient as before, in the background, while acting as a client device in the foreground. The multimodal patient monitor devices described herein are also configured to be worn by a patient, particularly when the device is operating in the patient monitor mode.

The example above, in which a nurse temporarily re-purposes a multimodal patient monitor being worn as a patient monitor to act as a client, the client device may function as a viewer. In client mode (which may also be referred to as viewer mode), the device receives notifications and data from the patient monitors of other patients within the wireless network, including patient alerts. In some embodiments, the clients may be location sensitive, so that only alerts or patient information for devices in the network that are relatively nearby (during nurse's bedside visit) would be presented, or fully presented (although an abbreviated and expandable presentation may be presented). For example, an alert may trigger only the client (viewer) within the nearest proximity to the patient in distress. In some embodiments, a client (viewer) may be carried by a caregiver such as a nurse. The client may be a dedicated client or viewer, or a multimodal device that is set to client mode.

The embodiment of the multimodal patient monitor 500 shown in FIG. 6 includes a wireless transmitter/receiver 511, a plurality of data inputs for receiving patient data 501, a controller (or “monitor controller”) 505 configured to control and configure the data inputs 501, a monitor processor 503 configured to process data received by the data inputs 501, a router processor 583 having processing resources for managing multiprotocol routing of packets received from the wireless receiver on the monitor processor 503, a network connector 593 configured to create a gateway between the mesh network and a second network, and a client processor 597 having resources for running client applications and for processing data received by the wireless receiver, or from the monitor 500 itself. The device of FIG. 6 may be otherwise similar to the embodiment of the dynamically reconfigurable monitor show in FIG. 1A.

The wireless transmitter/receiver 511 may be a single, integrated transmitter/receiver, or it may include a separate transmission channel and a receiving channel. The transmitter/receiver may also include a wireless controller 513 for controlling transmission and reception of information. This controller may also process information sent by the device, including formatting data (e.g., into packets, sentences, or any other appropriate format), adding error-correction codes, encryption, etc. These duties may also be performed, shared or controlled by the monitor processor 503. For example, the monitor processor may delegate processing of the data to be transmitted to the wireless controller 513. In some embodiments the wireless controller 513 is part of the monitor processor 503.

In some embodiments the wireless controller 513 is part of the router processor 583. The router processor may execute routing logic, including priority logic (e.g., priority comparison logic), and may store, buffer, and queue information for transmission by the device. As mentioned, the information passed between the nodes may be formatted in any appropriate manner, including as packets, words, etc. In some embodiments the router processor can also error-correct data (if sent with error correction codes). The router processor may allow the device 500 to act as a repeater node in the mesh network.

The client processor 597 may include resources (e.g., instructions, code, etc.) for running client applications allowing a device to act as a client in the network to view and/or interact with multiple patient monitors. In some embodiments the client processor 597 allows the device to interface with another device having a processor (e.g., a computer, PDA, etc.) and a presentation means (e.g., display, monitor, projector, speaker, etc.) so that the combination of the multimodal patient monitor acting in client mode and the other device can function as a client in the network. In some embodiments the client processor includes or is connected to sufficient resources (e.g., software, memory, hardware, presentation means, etc.) so that the multimodal device is able to act as a client without requiring an additional device having a processor.

Although many of the elements illustrated in FIG. 6 are shown as separate, any of these elements may be combined as parts of a single element (e.g., a single processor may operate as both a client processor 597 and a monitor processor 503).

The mode switch 599 may be an electronic switch (e.g., controlled by the monitor controller 505, which receives input from a client or server), or a manual switch (e.g., on the housing of the device), or both. Thus, the mode switch may allow the device to be toggled between modes, including combinations of modes. An indicator of the mode may also be included.

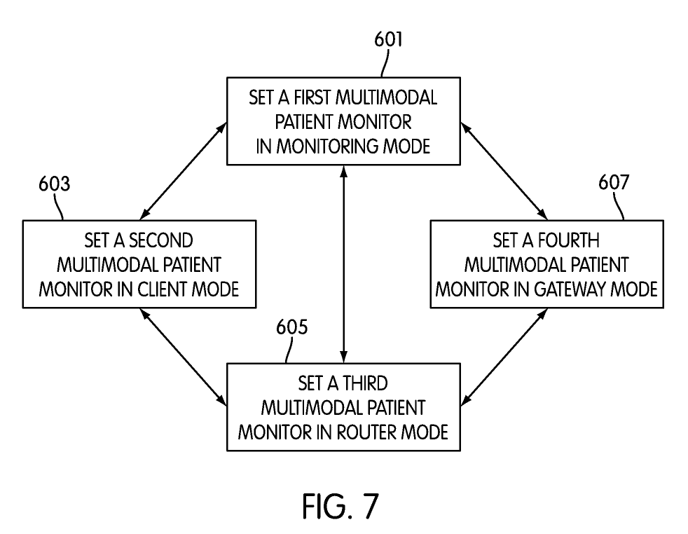

In operation, a multimodal patient monitor may be used to build a patient monitoring network for monitoring a plurality of patients, as illustrated in FIG. 7. For example, the device may be used to build a mesh network (e.g., an ad hoc wireless mesh network) of patient monitors by setting different multimodal devices to act as wearable patient monitors 601 (e.g., setting some devices to monitoring mode), and setting one or more devices to act as clients 603 (e.g., setting a device or devices into client mode). An additional multimodal device may be used as a gateway 607 (by toggling to gateway mode), or as a router 605 (by toggling to router mode). Thus, by providing a plurality of otherwise-identical multimodal devices (e.g., off the shelf), a complete network can be constructed. The size and complexity of the network is not limited.

Although a multimodal patient monitor may serve as a router, gateway, or other network element, and in some embodiments, it may not be necessary to include network elements other than the patient monitors themselves, in other embodiments, it may be desirable to include other dedicated network elements, such as repeaters and routers. For example, repeaters that are placed in fixed locations may increase the reliability of the network overall. One advantage of a multimodal patient monitor according to embodiments of the invention is that a dedicated repeater or router may have many of the same physical components as a multimodal patient monitor and much of the same software.

The invention described above can be useful for the tracking, monitoring, and management of patients during medical emergencies. It is often the case that in the chaotic and dynamic environment of a medical emergency, responders must care for an overwhelming number of casualties under severe resource limitations. Emergency response groups, such as fire, police, EMS, HazMat, hospitals, and public health groups, typically function as individual units, but must collaborate effectively during mass casualty events. Their effective response relies heavily on their ability to assess the rapidly changing needs of the on-going disaster by keeping an up-to-date triage count of the casualties. For years, responders performed triage of casualties using paper triage tags, clipboards of notes, and voice communications (over telephones and hand-held radios). Responders typically attach colored (e.g., red, yellow, green, black) paper triage tags to patients based upon assessed priority, and then report counts to Triage Officers over handheld radios. Triage Officers tally the patients on clipboards and report over handheld radios to Incident Commanders, who in turn request for the necessary resources (ambulances, supplies, responders). This workflow has proven labor intensive, time consuming, and prone to human error. As a result, medical emergency responders are plagued by a lack of accurate and timely information of their patient census: transport officers would call for transport with little information on how many casualties required transport, receiving hospitals would prepare beds for patients without knowledge of the number of incoming patient or their medical needs.

When used in emergency situations, patient data communication devices according to embodiments of the invention can configure themselves to adapt to evolving requirements and workflows without requiring the care providers, who may be of vastly different backgrounds and experience levels, to manually reconfigure them. Moreover, the reconfigurability and multimodal nature of the devices may reduce cost, because a single device or group of hardware elements may be used for multiple purposes. Finally, the mesh network may provide for reliable communications in situations in which communications network infrastructure may be very limited or nonexistent.

While the methods and devices have been described in some detail here by way of illustration and example, such illustration and example is for purposes of clarity of understanding only. It will be readily apparent to those of ordinary skill in the art in light of the teachings herein that certain changes and modifications may be made thereto without departing from the spirit and scope of the invention.

What is claimed is:

1. A patient information communication device, comprising:
one or more patient data inputs, each of the one or more patient data inputs being adapted to accept data;
one or more patient data outputs; and
a processor connected to the one or more patient data inputs and one or more patient data outputs, the processor being adapted: (1) to determine, based on a set of configuration instructions, which of the one or more patient data inputs and which of the one or more patient data outputs should be active, (2) to determine the manner and type of the data that should be outputted to the one or more patient data outputs, and (3) to accept new configuration instructions, either explicitly or as a result of a change in properties of the one or more patient data inputs, and reconfigure the patient information communication device based on the new configuration instructions;
wherein the patient information communication device is wearable.

2. The patient information communication device of claim 1, wherein the one or more data inputs are configured and adapted to receive data wirelessly, by direct manual entry on the device, or through a wired connection; and
wherein each of the one or more data inputs may be a wireless transceiver, an onboard user interface device, or an input/output port device.

3. The patient information communication device of claim 1, wherein the processor is further adapted (1) to process data generated actively or passively by one or more sources selected from the group consisting of a patient, a care provider, an external device, and a connected sensor or device, and (2) to process data indicative of one or more of patient conditions, environmental conditions, or conditions of other devices.

4. The patient information communication device of claim 3, wherein the connected sensor or device is selected from the group consisting of an image sensor, an ambient compound sensor, an ambient humidity sensor, an ambient temperature sensor, an ambient light sensor, and an ambient vibration sensor.

5. The patient information communication device of claim 3, wherein the connected sensor or device is selected from the group consisting of a pulse oximeter, an ECG, heart rate sensor, an EEG, a blood pressure sensor, a temperature sensor, a CO₂ sensor, a respiration sensor, a glucose sensor, a skin resistance sensor, an anemia detector, and a hydration sensor.

6. The patient information communication device of claim 3, wherein the connected sensor or device is a position sensor selected from the group consisting of a radio frequency ID sensor, a global positioning system transceiver, a gyroscope, and an accelerometer.

7. The patient information communication device of claim 1, further comprising a wireless transceiver unit, wherein the wireless transceiver unit serves as one of the patient data inputs and one of the patient data outputs.

8. The patient information communication device of claim 7, wherein the wireless transceiver unit is configured and adapted to allow the patient information communication device to act as a node of a network.

9. The patient information communication device of claim 8, wherein the network is a mesh network.

10. The patient information communication device of claim 9, wherein the mesh network allows the wireless transceiver unit to transmit and receive data by a direct link or by a multi-hop link to at least one destination.

11. The patient information communications device of claim 10, wherein the at least one destination comprises a single destination, multiple destinations, or an unspecified destination.

12. The patient information communication device of claim 10, wherein the device is configured and adapted to forward data from a second device through the mesh network to a destination specified by the data from the second device.

13. The patient information communication device of claim 7, wherein the wireless transceiver unit is further configured and adapted to receive new configuration instructions and to provide those instructions to the processor.

14. The patient information communication device of claim 1, further comprising a memory adapted to store configuration information for other devices, the device being adapted to provide the stored configuration information to the other devices.

15. The patient information communication device of claim 1, wherein at least one of the one or more patient data inputs is an annotation input adapted to receive annotation data.

16. The patient information communication device of claim 15, wherein the annotation data comprises one or more types of data selected from the group consisting of caregiver action data, date of caregiver action data, time of caregiver action data, patient vital sign data, patient priority data, nurse's notes, and data regarding the patient's chief complaint.

17. The patient information communication device of claim 1, further comprising a clock in communication with the processor, the processor being further adapted to time stamp the data.

18. The patient information communication device of claim 1, wherein one of the one or more patient data outputs is a display, and the processor is further adapted to determine which of the data will be output to the display.

19. The patient information communication device of claim 1, wherein the processor is further adapted to set an alarm or alert if at least some of the data is physiological data and at least some of that physiological data does not fall within specified parameters

20. A patient information communication device, comprising:
a wireless transceiver unit configured and adapted to input and output patient data; and
a processor connected to the wireless transceiver unit and adapted to configure the wireless transceiver unit to allow the patient information communication device to act as a node in a mesh network, the processor and wireless transceiver unit being operable to allow the device to perform one or more network node functions selected from the group consisting of data source, data forwarder, and data sink.

21. The patient information communication device of claim 20, wherein the processor is further adapted to allow the wireless transceiver unit to transmit and receive data associated with a single patient, to transmit and receive data associated with multiple patients, and to transmit and receive data not associated with any particular patient.

22. The patient information communication device of claim 20, wherein the patient information communication device is configured to perform one or more actions on data from other devices in the mesh network, the actions being selected from the group consisting of receiving, processing, and transmitting the data from other devices.

23. The patient information communication device of claim 22, wherein the patient information communication device accepts data received from the other devices in the mesh network for processing and transmission selectively, depending on the number of hops between the other patient information communication device and the patient information communication device.

24. A system for communicating and managing patient information, comprising:
a plurality of patient information communication devices, each of the patient information communication devices comprising:
a wireless transceiver unit configured and adapted to input and output patient data; and
a processor connected to the wireless transceiver unit and adapted to configure the wireless transceiver unit to allow the patient information communication device to act as a node in a mesh network, the processor and wireless transceiver unit being operable to allow the device to perform one or more network node functions selected from the group consisting of data source, data forwarder, and data sink,
at least some of the plurality of patient information communication devices being wearable;
wherein the plurality of patient information communication devices are configured to establish a mesh network with one another; and
wherein at least some of the plurality of patient information communication devices further comprise a memory, allowing those devices to perform one or more of storing data, forwarding data, or storing and forwarding data for others of the plurality of patient information communication devices.

25. The system of claim 24, wherein the processor is further adapted to determine whether data should be (1) outputted on the device or another device, (2) stored on the device or another device, and (3) processed on the device or another device.

26. The system of claim 24, wherein at least one of the plurality of patient information communication devices further comprises a user interface allowing the device to perform one or more of presenting, annotating, or modifying data from others of the plurality of patient information communication devices.

27. The system of claim 24, wherein the processor of at least one of the plurality of patient information communication devices is further adapted to process data for others of the plurality of patient information communication devices.

28. The system of claim 24, wherein the plurality of patient information communication devices is configured to assign a priority rank to each data packet transmitted through the mesh network and to transmit data with a higher priority rank before data with a lower priority rank.

29. The system of claim 17, further comprising a server configured and adapted to receive the data from the mesh network.
